(19) United States
(12) Patent Application Publication
(10) Pub. No.: US 2023/0000736 A1
(43) Pub. Date: Jan. 5, 2023

(54) COMPOSITIONS FOR SKINCARE AND USE THEREOF

(71) Applicant: ANJON BIOLOGICS, INC., Las Vegas, NV (US)

(72) Inventors: Michel Afargan, Raanana (IL); Raziel Ronen, Hod Hasharon (IL)

(73) Assignee: ANJON BIOLOGICS, INC., Las Vegas, NV (US)

(21) Appl. No.: 17/904,717

(22) PCT Filed: Feb. 8, 2021
(86) PCT No.: PCT/US2021/017099

§ 371 Date: Aug. 21, 2022

Related U.S. Application Data
(60) Provisional application No. 62/979,858, filed on Feb. 21, 2020.

(51) Int. Cl. A61K 8/14; A61K 8/23; A61K 8/27; A61Q 17/04; A61K 8/06

(52) U.S. Cl. CPC A61K 8/14; A61K 8/23; A61K 8/27; A61Q 17/04; A61K 8/062

(57) ABSTRACT

Compositions comprising an aqueous medium, lipids and nonionic surfactants and methods of their use in preventing skin damages, such as in subjects exposed to radiation, are provided.

### CROSS REFERENCE TO RELATED APPLICATION

This application claims priority to U.S. Provisional Application No. 62/979,858 filed on Feb. 21, 2020, which is incorporated herein by reference in its entirety.

### TECHNICAL FIELD

The present disclosure relates to formulations and use thereof in preventing skin damages, and the methods for preparing the formulations.

### BACKGROUND

Skin is one of the largest organs of the body. Skin is composed of two main layers: the surface epithelium or epidermis which includes the uppermost stratum corneum, and the subjacent connective tissue layer or dermis. Skin has several functions such as protecting the body from injury and dehydration, receiving environmental stimuli, excreting various substances, regulating body temperature, and helping to maintain water balance. Skin hydration is mostly linked to its water balance and water retention capacity and is important for maintaining the skin functions and health.

Radiation therapy has traditionally been the treatment of choice for locally or regionally advanced cancer. However, its therapeutic efficacy is often hindered by limited tolerance of normal tissues and by tumor radio-resistance. To improve therapeutic outcome, radiotherapy is frequently combined with chemotherapeutic drugs that may sensitize cells to radiation but are themselves cytotoxic. Skin damages due to radiation therapy represent the most frequent adverse effect of this life-saving treatment. In radiation therapy, the skin is exposed to extremely high energy radiation which exceeds its tolerability and protection capacity. Thus, the exposure of the skin to intense radiation energy causes severe dehydration and loss of its integrity. Radiation may cause severe burns of the skin and surrounding tissues as well as permanent changes in pigmentation. Consequently, the main clinical symptoms include skin redness, skin dryness, itching, blistering, peeling, and inflammation known as radiation dermatitis.

Further, the health and integrity of skin may be compromised by wounds, abrasions, ulcers, burns, infections, irritations, premature birth, and other conditions for which normal skin production and repair processes may be inadequate. Prolonged exposure to UV radiation, such as from the sun, can lead to the formation of light dermatoses and erythema, as well as increase the risk of skin cancers, such as melanoma, and accelerate skin aging, such as loss of skin elasticity and wrinkling.

Skin protection and treatments against skin damage encompass a variety of methods and products. These may range from protective skincare and pharmaceutical products, such as moisturizing creams, to symptomatic treatments, such as the use of topical anti-inflammatory compounds. Common remedies for skin protection contain a wide range of protective actives, including antioxidants, enzymes cofactors, humectants and other actives that may improve the healing of the damaged skin.

The key factor for the skin protection effect is the duration of action. Due to the properties of the skin, most topical skincare and pharmaceutical remedies are eliminated from the skin after a short period of time. This elimination is mainly due to their wash-off from the skin surface by water, and/or due to the rapid absorption of the product constituents followed by rapid clearance to deeper tissues and to the blood circulation. This inherent nature of the skin shortens the retention of the absorbed ingredients and/or water in the stratum corneum. Thus, the efficacy of most topically applied products only lasts for a relatively short period of time at upper skin layers, which makes it necessary to use and dose frequently.

A wide range of studies have been conducted to prolong the skin protection effect of topical formulations. These approaches have led to the development of many skincare and pharmaceutical products based on oil-in-water (o/w) and water-in-oil (w/o) emulsions/dispersions. In w/o dispersions, the water droplets are dispersed in the oily continuous phase. O/w dispersions contain dispersed oil constituents such as droplets or particles, for example lipid-based micelles or vesicles, that are dispersed in a continuous aqueous phase. In the case of micelles, the dispersed vesicles may take various types of morphologies, such as capsules, liposomes and niosomes. The vesicular systems increase water entrapment and can also be used as vehicles for effective delivery of active agents to the skin.

In order to prevent the rapid wash-out and clearance of water and ingredients from the skin, there is an unmet need for topical formulations to have prolonged retention at the stratum corneum. There is also an urgent need for compositions which can provide protection against skin damages such as from exposure to radiation as well as preventing skin cancer reoccurrence.

### SUMMARY

The present disclosure provides for a composition comprising an aqueous medium, lipids, and at least one nonionic surfactant. The lipids may comprise lipid-based vesicles dispersed in the aqueous medium. The lipid-based vesicles may be unilamellar and/or multilamellar and may have an aqueous core. In addition, the lipids may be dispersed in the cream matrix to form a part of a multilamellar matrix. The lipids may comprise at least an oil. The lipids may comprise at least one lipophilic component. The composition may comprise at least one trace element having a concentration ranging from about 0.0001% to about 0.1% by weight relative to the total weight of the composition. The trace element(s) may be water-soluble.

The present disclosure also provides for a composition comprising an aqueous medium, lipids, and at least one nonionic surfactant. The lipids may comprise lipid-based vesicles dispersed in the aqueous medium. The lipids may comprise at least one lipophilic component. The composition may comprise at least one trace element having a concentration ranging from about 0.015% to about 0.025% by weight relative to the total weight of the composition. The composition may comprise at least one trace element having a concentration ranging from about 0.018% to about 0.022% by weight relative to the total weight of the composition. The trace element(s) may be water-soluble.

The at least one trace element may be selenium (Se), zinc (Zn), or a combination thereof. Se or Zn may have an oxidative state ranging from −2 to +6. The composition may comprise about 0.02% selenium (Se) by weight relative to the total weight of the composition. The composition may further comprise about 0.0002% zinc (Zn) by weight relative to the total weight of the composition.

The composition may be substantially free of minerals other than Se and Zn.

The at least one nonionic surfactant may be a polyethoxylated saccharide derivative, a polyethoxylated sugar alcohol, a sugar fatty acid ester, a sugar-alcohol fatty acid ester, an emulsifying wax, a fatty alcohol, a pegylated lipid, a silicone oil, a silicone oil derivative, a glyceride, a polysaccharide, derivatives thereof, or combinations thereof.

The lipid-based vesicles may have a mean size ranging from about 0.1 micrometers to about 10 micrometers.

The composition may have a hydrophilic-lipophilic balance (HLB) value ranging from about 10 to about 14, or about 12.

The weight ratio of the lipids to the aqueous medium may be at least or about 1:1.5 or at least or about 1:1.7.

The weight ratio of the at least one nonionic surfactant to the lipids may be at least or about 1:1 or at least or about 1.5:2.

The composition may have a z-potential ranging from about 1 mV to about −60 mV, or from about −20 mV to about −40 mV.

The lipids may further comprise at least one wax.

The composition may comprise a multilamellar matrix.

The aqueous medium may have a pH ranging from about 5 to about 6.

The composition may further comprise an active agent. The active agent may be, but is not limited to, water-soluble (e.g., allantoin or any water-soluble active pharmaceutical agent), or lipophilic (e.g., one or more essential oils that may be dissolved in an alcohol such as ethanol, or any oil-soluble active pharmaceutical agent).

The active agent may be at a concentration ranging from about 0.1% to about 10% of the total weight of the composition.

The composition may further comprise at least one anionic surfactant. In one embodiment, the at least one anionic surfactant is a fatty acid salt, such as a stearic acid salt.

The present composition may be used for preventing or reducing damage to the skin of a subject undergoing (or have undergone or will undergo) radiotherapy or laser treatment. The present composition may be used for preventing or reducing skin cancer occurrence or reoccurrence.

The present disclosure provides for a method of preventing or reducing skin damage in a subject in need thereof. The method may comprise topically applying to the skin of the subject an effective amount of the present composition.

The skin damage may be caused by exposure to radiation or laser.

The subject may be undergoing radiotherapy, has undergone radiotherapy, or will undergo radiotherapy.

The composition may be applied to the skin of the subject prior to, during or after, radiotherapy.

In one embodiment, the radiotherapy may be external-beam radiation therapy.

The present disclosure provides for a process for preparing a topical composition. The process may comprise:
(a) heating lipids and oil-soluble ingredients at a temperature ranging from about 65° C. to about 85° C. to obtain an oil phase;
(b) heating water-soluble ingredients in water at a temperature ranging from about 65° C. to about 85° C. to obtain an aqueous phase;
(c) emulsifying the oil phase in the aqueous phase for a period of time to obtain a liquid emulsion;
(d) cooling the liquid emulsion to a temperature ranging from about 25° C. to about 50° C. to obtain a semi-solid emulsion;
(e) adding a solution comprising at least one trace element to the semi-solid emulsion at a temperature ranging from about 30° C. to about 50° C. to obtain a mixture; and
(f) adjusting a pH of the mixture to about 4-6, if the mixture's pH is not 4-6, to obtain the topical composition.

In one embodiment, in step (a), the heating may be at about 75° C.

In certain embodiments, in step (a), the oil phase may comprise beeswax, cetearyl alcohol, cetyl alcohol, glyceryl stearate, isopropyl myristate, paraffin oil, mineral oil, sesame oil, shea butter and sorbitan tristearate, a polyethylene glycol (PEG) ester of stearic acid, or combinations thereof.

In certain embodiments, in step (b), the aqueous phase may comprise glycerin, dimethicone, allantoin, cetearyl alcohol, PEG-20 stearate, Sabowax Fl-20, polysorbate 20, polysorbate 80, or combinations thereof.

In one embodiment, in step (b) the heating is at about 75° C.

In one embodiment, in step (c) the emulsifying is by homogenization.

In one embodiment, in step (c) the period of time is about 45 minutes.

In one embodiment, in step (d) the cooling is conducted under continuous agitation.

The process may further comprise step (g) of mixing a solution comprising at least one preservative and at least one antioxidant to the semi-solid emulsion after step (d).

The at least one preservative may comprise imidazolidinyl urea, methylparaben, propylparaben, 1,3-Dimethylol-5,5-dimethyl hydantoin (DMDMH), butylated hydroxytoluene (BHT), or combinations thereof.

In one embodiment, in step (g) the mixing is conducted at about 50° C.

In one embodiment, in step (e) the adding is conducted at about 40° C.

In one embodiment, the solution comprising at least one trace element has a pH of about pH3-4.

In one embodiment, in step (f) the pH of the mixture is adjusted to about pH5-5.5.

### BRIEF DESCRIPTION OF THE DRAWINGS

FIG. 1A shows the cryo-scanning electron microscopy (cryo-SEM) image of the dispersed lipid-based vesicles in an embodiment of the present composition.

FIG. 1B shows the confocal (bright field) image of the dispersed lipid-based vesicles in an embodiment of the present composition.

FIG. 2 is a cryo-SEM image showing the lamellar texture of the cream matrix of an embodiment of the present composition.

FIG. 3A is a cryo-SEM image showing the multilamellar lipid-based vesicle as an example of the coacervation induced by selenium (Se) and zinc (Zn).

FIG. 3B is a cryo-SEM image showing the lamellar lipid-based vesicles in the absence of trace elements.

FIG. 3C is a cryo-SEM image showing an example of the lamellar lipid-based vesicles of an embodiment of the present composition produced with a mineral extract but without selenium (Se) and zinc (Zn).

FIG. 3D is a cryo-SEM image of an embodiment of present composition with the trace elements selenium (Se) and zinc (Zn).

FIG. 3E is a cryo-SEM image of an embodiment of present composition with no minerals.

FIG. 3F is a cryo-SEM image of an embodiment of present composition with a mineral extract but without Se/Zn.

FIG. 4A is a confocal microscopy image showing an embodiment of the present composition with the minerals extract but without Se/Zn.

FIG. 4B is a confocal microscopy image showing an embodiment of the present composition with selenium (Se) and zinc (Zn).

FIG. 4C is a confocal microscopy image (Nile red) showing an embodiment of the present composition with a mineral extract but without Se/Zn.

FIG. 4D is a confocal microscopy image (Nile red) showing an embodiment of the present composition with selenium (Se) and zinc (Zn).

FIG. 5A shows the z-potential of the mineral extract in an embodiment of the present composition (without Se/Zn).

FIG. 5B shows the z-potential of the mineral extract in an embodiment of the present composition with Se/Zn only.

FIGS. 6A, 6B and 6C show very poor dispersion of particles. This example indicates negligible effect of trace elements on the oil constituents.

FIGS. 7A and 7B show very poor emulsification of water in oil by Tween 80.

FIGS. 8A, 8B and 8C show a significant dispersion of droplets in comparison to Examples 9 and 10.

FIG. 9 indicates that selenium (Se) and zinc (Zn) increased the emulsification of the surfactant.

FIG. 10 is a photo showing that the semi-solid texture of the cream base with no indication of reduced viscosity nor any disruption of the cream base consistency.

FIG. 11A is a photo depicting the damaged skin of a female patient with breast cancer treated with radiation therapy. The marked area in FIG. 11A depicts the erythema and desquamation foci at the site of radiation application.

FIG. 11B is a photo depicting the same patient of FIG. 11A after 1 week of daily treatment with the present composition which was applied topically. The marked area in FIG. 11B shows significant recovery of the skin damage after a 1-week treatment period.

FIG. 11C is a photo depicting the damaged skin of another female patient having breast cancer treated with radiation therapy. The marked area in FIG. 11C depicts dark skin pigmentation—no desquamation using of the present composition from the beginning of radiation therapy. Only light pigmentation change observed at this early stage of the treatment course.

FIG. 11D is a photo depicting the same patient of FIG. 11C after 4-week of radiation therapy cycles treated daily by an embodiment of the present composition. The marked area in FIG. 11D depicts significant healthy skin with no indication of skin damage, nor pigmentation or dryness.

FIG. 12A shows the morphology of the composition with the extracted minerals (Example 1) but without selenium (Se) and zinc (Zn).

FIG. 12B shows an enlarged portion of FIG. 12A.

FIG. 12C shows the morphology of an embodiment of the present composition with only selenium (Se) and zinc (Zn) as described in Example 2.

FIG. 12D shows an enlarged portion of FIG. 12C.

FIG. 12E shows the morphology of a composition with only selenium (Se) and zinc (Zn) as described in Example 2, although the concentration of the trace elements was increased to 0.006%.

FIG. 12F shows an enlarged portion of FIG. 12E.

FIG. 12G shows more examples of the morphology of the composition with 0.06% selenium (Se) and zinc (Zn).

FIG. 12H shows more examples of the cross-section morphology of a composition with 0.06% selenium (Se) and zinc (Zn).

FIG. 12I shows the effect of reduced selenium (Se) and zinc (Zn) concentration by 50% (to 0.01wt %) of a composition.

FIG. 12J shows an enlarged portion of FIG. 12I.

FIG. 13A shows the efficacy of Tween 80 as a nonionic surfactant in an oil-in-water emulsion 1-hour post emulsification.

FIG. 13B shows the effect of other minerals on the emulsification capacity of FIG. 13A after vigorous stirring.

FIG. 13C shows additional comparative assessments of the physical effect of selenium (Se) and zinc (Zn) and their concentrations of nonionic surfactants-based emulsion.

FIG. 14 shows oil-in-water basic emulsions emulsified with the nonionic surfactant Tween-80 as an exemplary surfactant.

FIG. 15A shows the sun protection factor (SPF) data of an SPF-15 sunscreen which was used as a positive control.

FIG. 15B shows the SPF data of Group lb, an embodiment of the present composition.

FIG. 16 is a photo of the tested 6-well plate with human skin grafts cultured in the growth medium as evaluated in the photoprotection study.

FIG. 17 shows the comparative photoprotection results of the epidermal viability of the human skin grafts treated with an embodiment of the present composition (ARC) compared to an SPF-15 sunscreen (a positive control) under the same experimental condition.

FIG. 18 shows the comparative photoprotection results of interleukin 8 (IL-8) of the human skin grafts treated with an embodiment of the present composition (ARC) compared to an SPF-15 sunscreen (a positive control) under the same experimental conditions.

### DETAILED DESCRIPTION

The present disclosure relates to compositions containing an aqueous dispersion of lipid-based vesicles. The compositions have improved thermodynamic stability. The lipid phase and the lipid-based vesicles in the composition may contain oils, waxes and surfactants which are characterized by lamellar structures having one or more lipid layers separated from each other by aqueous layers. The lipid-based vesicles may have aqueous cores. Also encompassed by the present disclosure are methods of preparing the compositions and methods of preventing or reducing skin damage in a subject using the composition.

The present disclosure provides a composition for skin protection where the composition exhibits significantly prolonged retention of water and other ingredients in the skin. The composition simulates the stratum corneum structure by dispersed, lamellar lipid-based vesicles in a continuous water phase. The morphology and size of the vesicles enable their prolonged retention in the skin and effective entrapment and delivery of a significant water and other ingredient payload to the upper layers of the skin for a prolonged time. Following the topical application of the composition, it is rapidly absorbed and protects the skin against damages, e.g., induced by radiation therapy. Notably, washing the skin with water does not interfere with the function of the composition due to its rapid absorption. More specifically, the aqueous dispersion of lamellar lipid-based vesicles exhibits significant retention in the stratum corneum which provides effective skin protection even with once daily application.

The composition provides better hydration and bioavailability of active agents at the upper layers of the skin. By achieving this retention profile, the present composition provides effective and prolonged skin protection against dehydration and loss of integrity as well as a vehicle for effective delivery of active agents to the stratum corneum.

The present disclosure provides for a composition comprising an aqueous medium, lipids, and at least one nonionic surfactant. The lipids may form/comprise lipid-based vesicles dispersed in the aqueous medium. The lipid-based vesicles may be unilamellar and/or multilamellar and have an aqueous core. The lipids may comprise at least an oil.

Oils may include, but are not limited to, vegetable oils (e.g., refined vegetable oils), butters, and mineral oils. Non-limiting examples of vegetable oils include, safflower oil, rice bran oil, olive oil, soybean oil, sesame oil, coconut oil, peanut oil, corn oil, sunflower oil, canola oil, grapeseed oil, avocado oil, cottonseed oil, walnut oil, palm oil, and almond oil. Non-limiting examples of butters include, shea butter, cocoa butter, almond butter, and peanut butter. Non-limiting examples of mineral oils include, paraffin oil, silicone oils (e.g., polysiloxanes).

The lipids may further comprise at least one wax. The wax(es) may act as a thickener for the semi-solid composition of lipid-based vesicles dispersed in a water-in-oil cream base.

Non-limiting examples of waxes include, beeswax, jojoba wax, candelilla wax and petrolatum.

The composition may comprise a multilamellar matrix.

The composition may comprise at least one trace element having a concentration ranging from about 0.0001% to about 0.1%, from about 0.0002% to about 0.09%, from about 0.0004% to about 0.08%, from about 0.0006% to about 0.07%, from about 0.0008% to about 0.06%, from about 0.001% to about 0.05%, from about 0.002% to about 0.04%, from about 0.004% to about 0.03%, from about 0.006% to about 0.02%, from about 0.008% to about 0.02%, from about 0.01% to about 0.02%, from about 0.0005% to about 0.09%, from about 0.001% to about 0.08%, from about 0.001% to about 0.07%, from about 0.001% to about 0.0.06%, from about 0.001% to about 0.05%, from about 0.001% to about 0.04%, from about 0.001% to about 0.03%, from about 0.001% to about 0.02%, from about 0.002% to about 0.03%, from about 0.002% to about 0.02%, from about 0.002% to about 0.025%, from about 0.01% to about 0.03%, from about 0.02% to about 0.03%, from about 0.015% to about 0.025%, from about 0.018% to about 0.022%, about 0.002%, about 0.018%, or about 0.02%, by weight relative to the total weight of the composition.

In certain embodiments, the at least one trace element is selenium (Se), zinc (Zn), or a combination thereof. Se or Zn may have an oxidative state ranging from −2 to +6.

In one embodiment, the composition may comprise about 0.02% selenium (Se) by weight relative to the total weight of the composition. In one embodiment, the composition may further comprise about 0.002% comprising zinc (Zn) by weight relative to the total weight of the composition.

In some embodiments, selenium (Se), and zinc (Zn) have a weight ratio of about 200:1 to about 100:1, about 150:1 to about 50:1, about 150:1 to about 100:1, about 100:1 to about 50:1, or about 100:1 to about 10:1. In some embodiments, selenium (Se), and zinc (Zn) have a weight ratio of at least or about 150:1, at least or about 120:1, at least or about 100:1, at least or about 95:1, at least or about 90:1, at least or about 85:1, at least or about 80:1, at least or about 75:1, at least or about 70:1, at least or about 65:1, at least or about 60:1, at least or about 55:1, or at least or about 50:1. In some embodiments, selenium (Se), and zinc (Zn) may have a weight ratio of 10:1 to 1:1. In some embodiments, selenium (Se) and zinc (Zn) may have a weight ratio of 9:1 to 1:1. In some embodiments, selenium (Se) and zinc (Zn) have a weight ratio of 8:1 to 1:1. In some embodiments, selenium (Se) and zinc (Zn) have a weight ratio of 8:1 to 2:1. In some embodiments, selenium (Se) and zinc (Zn) have a weight ratio of 5:1 to 1:1. In some embodiments, selenium (Se) and zinc (Zn) have a weight ratio of 6:1 to 2:1. In some embodiments, selenium (Se) and zinc (Zn) have a weight ratio of 6:1 to 3:1.

In certain embodiments, the composition is substantially free of minerals other than Se and Zn.

The composition may or may not further comprise one or more minerals selected from potassium (K), calcium (Ca), magnesium (Mg), sulfate (SO₄), bromide (Br), manganese (Mn), copper (Cu), sulfur (S), silica (SiO₂), iron (Fe), bicarbonate, tellurium (Te), sodium (Na) and chloride (Cl), and derivatives thereof. As used herein the term “derivatives” refers to oxidized forms of the mineral, solid solutions of the mineral, organic composition of the mineral, inorganic composition of the mineral, or other derivatives. As a non-limiting example, ZnO is an oxidized derivative of zinc. As another non-limiting example, selenium methionine is an organic composition of selenium.

The nonionic surfactant may be a polyethoxylated saccharide derivative, a polyethoxylated sugar alcohol, a sugar fatty acid ester, a sugar-alcohol fatty acid ester, an emulsifying wax, a fatty alcohol, a pegylated lipid, a silicone oil, a silicone oil derivative, a glyceride, a polysaccharide, derivatives thereof, or combinations thereof.

In certain embodiments, non-ionic surfactants include, but are not limited to, the reaction products of an aliphatic alcohol or alkylphenol (e.g., having 6 to 20 carbon atoms in a linear or branched alkyl chain) with an alkylene oxide (e.g., ethylene oxide, and/or propylene oxide). The alkylene oxide may be from about 6 moles to about 60 moles per mole of the aliphatic alcohol or alkylphenol. In certain embodiments, non-ionic surfactants may include alkylamine oxides, mono- and di-alkylalkanolamides, fatty acid esters of polyethylene glycols, ethoxylated fatty acid amides, saturated fatty acid alcohols reacted with ethylene oxide, alkyl polyglycosides, and sorbitan ether esters. In some embodiments, the non-ionic surfactant may be ceteareth-2, ceteareth-3, ceteareth-4, ceteareth-5, ceteareth-6, ceteareth-7, ceteareth-8, ceteareth-9, ceteareth-10, ceteareth-11, ceteareth-12, ceteareth-13, ceteareth-14, ceteareth-15, ceteareth-16, ceteareth-17, ceteareth-18, ceteareth-20, ceteareth-22, ceteareth-23, ceteareth-24, ceteareth-25, ceteareth-27, ceteareth-28, ceteareth-29, ceteareth-30, ceteareth-33, ceteareth-34, ceteareth-40, ceteareth-50, ceteareth-55, ceteareth-60, ceteareth-80, ceteareth-100, and the like or combinations thereof, or one or more ceteareths in combination with a fatty acid alcohol such as stearyl alcohol, oleyl alcohol, linoleyl alcohol, arachidyl alcohol, cetyl alcohol, and the like.

The composition may comprise one or more nonionic surfactants having a concentration ranging from about 5% to about 30%, from about 5% to about 25%, from about 5% to about 20%, from about 10% to about 20%, from about 5% to about 10%, from about 5% to about 15%, from about 15% to about 25%, from about 15% to about 20%, from about 10% to about 25%, from about 15% to about 30%, from about 20% to about 30%, from about 20% to about 25%, from about 0.1% to about 20%, from about 0.5% to about 20%, about 22%, about 18%, or about 20%, by weight relative to the total weight of the composition.

The lipid-based vesicles may have a mean size ranging from about 0.1 micrometers to about 10 micrometers, from about 0.1 micrometers to about 5 micrometers, from about 0.2 micrometers to about 5 micrometers, from about 0.5 micrometers to about 5 micrometers, from about 1 micrometer to about 10 micrometers, from about 1 micrometer to about 5 micrometers, about 3.5 micrometers, or about 5 micrometers.

In certain embodiments, in the composition, the weight ratio of the lipids to the aqueous medium may range from about 1:1 to about 1:3, from about 1:1 to about 1:2, from about 1:1 to about 1:1.8, from about 1:1 to about 1:1.7, from about 1:1 to about 1:1.5, from about 1:1.5 to about 1:1.7, about 1:1.5, or about 1:1.7.

In certain embodiments, in the composition, the weight ratio of the nonionic surfactant(s) to the lipids may range from about 1:1 to about 1:3, from about 1:1 to about 1:2, from about 1:1 to about 1:1.8, from about 1:1 to about 1:1.7, from about 1:1 to about 1:1.5, from about 1:1 to about 1:1.4, from about 1:1 to about 1:1.3, about 1:1, or about 1.5:2.

The composition may have a hydrophilic-lipophilic balance (HLB) value ranging from about 10 to about 14, from about 11 to about 14, from about 12 to about 14, from about 12 to about 13, from about 10 to about 13, or about 12. If the composition has two or more surfactants or oils, its HLB values may be the weighted average of the HLB values for each component.

The composition may have a z-potential ranging from about −60 mV to about 1 mV, from about −50 mV to about 1 mV, from about −40 mV to about −10 mV, from about −40 mV to about −20 mV, from about −50 mV to about −20 mV, from about −50 mV to about −10 mV, or from about −30 mV to about −20 mV.

The composition or the aqueous medium in the composition may have a pH ranging from about 5 to about 6. The composition (or the aqueous medium in the composition) may have a pH of less than 7, a pH in the range of 5-7, a pH in the range of 5-6.5, a pH in the range of 5-6, a pH in the range of 5-5.5, or about pH 5.5. The composition (or the aqueous medium in the composition) may have a pH of less than 8, a pH in the range of 5-8, a pH in the range of 1-8, a pH in the range of 1-6, or a pH in the range of 2-6. In certain embodiments, the pH of the composition (or the aqueous medium in the composition) may be a neutral to mildly acidic pH. For example, in various embodiments, the pH of the composition (or the aqueous medium in the composition) may be from about 2.5 to about 7.0, from about 4.0 to about 7.0, or from about 4.0 to about 5.5. In other embodiments, the pH of such composition (or the aqueous medium in the composition) may be about 4.0 to about 5.0. In other embodiments, the pH of the composition (or the aqueous medium in the composition) may be about 4.0 to about 8.0.

The composition may comprise an active agent. The active agent may be water-soluble (e.g., allantoin), or lipophilic (e.g., essential oils dissolved in an alcohol such as ethanol). The active agent may be at a concentration ranging from about 0.01% to about 5%, from about 0.1% to about 1%, from about 1% to about 10%, about 5%, of the total weight of the composition. In one embodiment, the composition contains 4-5% essential oils and 15-16% ethanol of the total weight of the composition. In one embodiment, essential oils comprise lavender oil, eucalyptus oil and clove oil at a weight ratio of 1:1:1 dissolved in ethanol.

The composition may further comprise at least one anionic surfactant. The anionic surfactant may serve as a negative charge booster of the negative charge of the dispersive lipid-based vesicles. The anionic surfactant may be a fatty acid salt, such as a stearic acid salt, or a mono- or di-ester of fatty acids and glycerol. The fatty acid salt may be a monovalent salt, such as a potassium or sodium salt. The anionic surfactant may have a concentration ranging from about 0.5% to about 5%, about 0.5% to about 4%, about 0.5% to about 3%, about 0.5% to about 2%, about 1% to about 5%, about 1% to about 4%, about 1% to about 3%, or about 1% to about 2%, of the total weight of the composition.

The present disclosure also relates to compositions and methods of production of stable oil-in-water emulsions comprising a relatively high lipid phase (e.g., about 40% by weight of the composition). In one embodiment, for every gram of the composition, there are 0.4 grams of lipids (e.g., one or more oils and/or one or more waxes) and at least one surfactant.

In certain embodiments, the lipid phase comprises oils (and/or waxes) and nonionic surfactants, e.g., in a weight ratio of about 1:1. The lipid phase is dispersed in the aqueous phase (e.g., about 60% by weight of the composition). In one embodiment, for every gram of the composition, there are 0.6 grams of aqueous medium.

The present compositions may contain one or more trace elements and/or salts thereof. The trace element may be water-soluble. The trace elements may affect the lamellar structure of the cream matrix (or the composition) and/or of the dispersed lipid-based vesicles. In certain embodiments, in an oil-in-water emulsion having a nonionic surfactant, the specific types of the trace elements at specific concentrations, combined with the method of formulation, may enable the transition of the dispersed unilamellar lipid vesicles to multilamellar. In addition, the trace elements may induce a multilamellar morphology and order of the lamellas of the cream matrix which thus increases the viscosity of the cream base. The multilamellar texture of the cream bases (or the composition) significantly increases its water load which prolongs water retention in the skin and acts as effective vehicles for the delivery of water-soluble and/or lipophilic active agents to the skin.

In certain embodiments, the specific trace elements and their specific concentrations ranges enable a controlled coacervation of the lipid phase in the oil-in-water emulsion system. The lipid phase may contain a mixture of oils, wax and nonionic surfactants. The coacervation can improve the thermodynamic stability of the dispersed lipid vesicles of the end-product, the cream base (or the composition).

The trace elements include, but are not limited to, selenium (Se) and zinc (Zn). Selenium (Se) and zinc (Zn) are amphoteric trace elements. They may exist in the water phase as either cations or anions, with their valency ranging from −2 to +6. The trace elements may also include a trace element that have an oxidative state similar to those of selenium (Se) and zinc (Zn), and a trace element that can act as an effective coacervation-inducing agent in a nonionic surfactant-based emulsion.

In one embodiment, the present composition contains an oil-in-water emulsion that comprises about 20% lipids/oils and about 60% of an aqueous medium emulsified with a relatively high concentration (about 15%) of surfactants. The relatively high surfactant concentration enables the compatibility of trace elements with the dispersion constituents. Moreover, it enables a controlled coacervation of the dispersed lipid-based vesicles and their transition from unilamellar to multilamellar nano-sized and/or micro-sized capsules. The trace elements induce controlled coacervation to produce dispersed multilamellar lipid-based vesicles in a nonionic surfactant-based, water-in-oil emulsion of a cream base having a lamellar matrix. The process of making the present composition having a lamellar vesicular system is relatively simple compared to the known methods in the art of particle sciences such as the methods to make niosomes.

The microscopic morphology and physical properties of the lipid-based vesicles and the microscopic texture of the cream bases have been studied in detail. The composition has a texture which simulates the naturally occurring, lipid-rich skin protectant of infants known as Vernix Caseosa. Therefore, we termed our novel multilamellar lipid-based vesicles “Vernisomes”.

The present disclosure provides skin protective compositions and methods of production of aqueous colloidal cream bases by controlled trace elements mediated coacervation for skincare and pharmaceutical use.

In one embodiment, the composition is based, in part, on the surprising finding of a unique composition of trace elements induced coacervated multilamellar lipid-based vesicles dispersed in the cream base useful for preventing damage to the skin, including, but not limited to, burns and cancer such as in subjects exposed to radiation. Without limiting the invention to a particular theory or mechanism of action, the compositions described herein may act by allowing the penetration of radiation through the skin (e.g., the epidermis) to the targeted site rather than partially reflecting off the skin and causing damage.

According to one embodiment, the present composition comprises about 20 wt % of oils emulsified by at least 15 wt % of a mixture of nonionic surfactants in a continuous aqueous phase of about 60 wt %.

According to one embodiment, the present composition comprises a stable oil-in-water emulsion.

According to one embodiment, the present composition comprises a stable oil-in-water emulsion with an HLB value of about 9-13, 10-14, 10 or 12.

According to one embodiment, the present composition comprises lamellar lipid-based vesicles dispersed in an aqueous phase.

According to one embodiment, the present composition comprises small unilamellar lipid-based vesicles dispersed in a continuous aqueous phase.

According to one embodiment, the present composition comprises lamellar lipid-base vesicles (e.g., unilamellar vesicles) dispersed in an aqueous phase where the vesicles have a mean size ranging from about 0.1 micrometers to about 10 micrometers, from about 0.1 micrometers to about 5 micrometers, or about 3.5 micrometers.

According to one embodiment, the present composition comprises lamellar lipid-base vesicles (e.g., unilamellar vesicles) dispersed in an aqueous phase where the vesicles have a mean size of about 3.5 micrometers.

According to one embodiment, the present composition has a z-potential range of −30 mV to −40 mV, or −38 mV.

According to one embodiment, the present composition comprises 0.02 wt % selenium (Se) and zinc (Zn). The solution containing Se and Zn is added to the cream base at a weight ratio of 1:10. In one embodiment, for every gram of the composition, 0.1 grams of the solution containing Se and Zn is added. In some embodiments, the weight ratio of the solution containing Se/Zn to the composition ranges from about 1:5 to about 1:50, about 1:5 to about 1:40, about 1:5 to about 1:30, about 1:5 to about 1:20, or about 1:5 to about 1:10. In some embodiments, the weight ratio of the solution containing Se/Zn to the composition is at least or about 1:5, at least or about 1:6, at least or about 1:7, at least or about 1:8, at least or about 1:9, at least or about 1:10, at least or about 1:12, at least or about 1:15, at least or about 1:20, at least or about 1:25, at least or about 1:30, at least or about 1:35, or at least or about 1:40.

According to one embodiment, the final content of selenium (Se) in the composition is about 0.018% by weight and zinc (Zn) of about 0.00018% by weight. In one embodiment, the present composition contains about 0.02% by weight of selenium (Se) and about 0.0002% by weight of zinc (Zn).

According to one embodiment, the mixture of the trace elements selenium (Se) and zinc (Zn) increases the viscosity of the dispersion system. According to another embodiment, the mixture of the trace elements selenium (Se) and zinc (Zn) increases the viscosity of the dispersion system by coacervation.

According to one embodiment, the mixture of the trace elements selenium (Se) and zinc (Zn) induces controlled coacervation of the dispersion system.

According to one embodiment, the mixture of the trace elements selenium (Se) and zinc (Zn) induces a controlled coacervation of the dispersion system by modifying the morphology of the cream matrix and of the dispersed lamellar lipid-based vesicles into multilamellar lipid-based vesicles.

According to one embodiment, the present composition comprises multilamellar lipid-based vesicles that are stably dispersed in an oil-in-water cream base.

According to one embodiment, the present composition comprises multilamellar lipid-based vesicles that are stably dispersed in an oil-in-water cream base which have an HLB value of 10-14, or 12.

According to one embodiment, the present composition comprises multilamellar lipid-based vesicles that are stably dispersed in an oil-in-water cream base and have a size range of 0.1-10 micrometers, 1-5 micrometers, or 5 micrometers.

According to one embodiment, the present composition comprises multilamellar lipid-based vesicles that are stably dispersed in an oil-in-water cream base and have a z-potential of −20 to −40 mV, −20 to −30 mV, or −23 to −37 mV.

According to one embodiment, the mixture of the trace elements selenium (Se) and zinc (Zn) induces a controlled coacervation of the dispersion system by incremental reduction of the z-potential from −40 to −20 mV of the dispersion system.

According to one embodiment, the present composition comprises a stable oil-in-water emulsion of trace elements which induce coacervation and dispersed multilamellar lipid-based vesicles for the encapsulation and delivery of an active agent (e.g., water-soluble active agents such as allantoin, or lipophilic active agents such as essential oils dissolved in ethanol) to skin. The concentration of the active agent may be about 5 wt %. The present compositions have enhanced skin protective function against skin damage.

According to one embodiment, the present composition comprises a stable oil-in-water emulsion of trace elements which induce coacervation and dispersed multilamellar lipid-based vesicles for the entrapment and delivery of water to the skin.

According to one embodiment, the present composition comprises a stable oil-in-water emulsion of trace elements which induce coacervation and dispersed multilamellar lipid-based vesicles for the entrapment of about 60 wt % of water and delivery of water to the skin.

In certain embodiments, on the microscopic and macroscopic levels, coacervation of the dispersed oils and nonionic surfactants results in uniform dispersion of multilamellar lipid vesicles in the water phase of the present compositions (or in the matrix of the cream base where the lipid vesicles act as thickeners for the cream base). The multilamellar vesicles enhance the cream base stability and prolong the retention of the formulation constituents on 20 the skin.

The present disclosure provides compositions and methods of production of skin-protective compositions which comprise dispersed multilamellar vesicles in a lamellar cream matrix. The lamellar lipid-based vesicles and the lamellar matrix of the composition can deliver high entrapped water payload and active agents with prolonged retention capacity that enables significant protection against radiation-induced skin damage.

The present disclosure provides a method for screening a wide range of minerals to identify and select trace elements that can induce the coacervation effects as described herein.

The present composition may be used for preventing or reducing damage to the skin of a subject undergoing radiotherapy or laser treatment, and/or for preventing or reducing skin cancer occurrence or reoccurrence.

The present disclosure provides for a method of preventing or reducing skin damage in a subject in need thereof. The method may comprise topically applying to the skin of the subject an effective amount of the present composition.

The skin damage may be caused by exposure to radiation or laser. The radiotherapy may be external-beam radiation therapy.

The subject may be undergoing or may have undergone radiotherapy. In certain embodiments, the subject will undergo radiotherapy. The composition may be applied to the skin of the subject prior to radiotherapy.

In some embodiments, the compositions and methods described herein are useful for prophylaxis of skin damages due to radiation or laser treatments. In some embodiments, the compositions and methods described herein are useful for prophylaxis of skin cancer.

Also encompassed by the present disclosure is a process for preparing a topical composition. The process may comprise:
(a) heating lipids and oil-soluble ingredients at a temperature ranging from about 65° C. to about 85° C. (e.g., about 75° C. or 80° C.) to obtain an oil phase;
(b) heating water-soluble ingredients in water at a temperature ranging from about 65° C. to about 85° C. (e.g., about 75° C. or 80° C.) to obtain an aqueous phase;
(c) emulsifying (e.g., by homogenization) the oil phase in the aqueous phase for a period of time (e.g., from about 20 minutes to about 2 hours, from about 30 minutes to about 1.5 hours, from about 30 minutes to about 1 hour, from about 30 minutes to about 50 minutes, or about 45 minutes) to obtain a liquid emulsion;
(d) cooling the liquid emulsion to a temperature ranging from about 25° C. to about 50° C. to obtain a semi-solid emulsion (e.g., under continuous agitation);
(e) adding a solution comprising at least one trace element to the semi-solid emulsion at a temperature ranging from about 30° C. to about 50° C. (e.g., about 40° C.) to obtain a mixture;
(f) adjusting a pH of the mixture to about 4-6 (or about 5-5.5), if the mixture's pH is not 4-6 (or about 5-5.5), to obtain the topical composition.

The process may further comprise step (g) of mixing a solution comprising at least one preservative and at least one antioxidant to the semi-solid emulsion after step (d) at a temperature ranging from about 30° C. to about 70° C. (e.g., about 50° C.). The at least one preservative may comprise imidazolidinyl urea, methylparaben, propylparaben, 1,3-Dimethylol-5,5-dimethyl hydantoin (DMDMH), butylated hydroxytoluene (BHT), or combinations thereof.

The oil phase may comprise beeswax, cetearyl alcohol, cetyl alcohol, glyceryl stearate, isopropyl myristate, paraffin oil, mineral oil, sesame oil, shea butter, sorbitan tristearate, a denatured alcohol (e.g., Alcohol SD 40), a polyethylene glycol (PEG) ester of stearic acid (e.g., PEG 100 stearate), the salts thereof, or combinations thereof. For example, the salts may be potassium or sodium salts. The salts may be potassium stearate or sodium stearate.

The aqueous phase may comprise at least one trace element, glycerin, dimethicone, allantoin, cetearyl alcohol, PEG-20 stearate (e.g., Sabowax FL-20 is a blend of cetearyl alcohol and PEG-20 stearate), Sabowax Fl-20, polysorbate 20, polysorbate 80, alcohol, at least one water-soluble preservative (such as methyl and propyl parabens, triethanolamine), or combinations thereof.

In step (e), the solution comprising at least one trace element may have a pH of about 3-4 or about 3-3.5.

### Conditions to be Treated

The composition may be formulated as a topical composition. The composition may be in the form of a cream, an ointment, a gel, lotion, liniment, paste or an emulsion.

Radiation therapy associated with anti-cancer treatments may result in skin dehydration and damages (radiation dermatitis). The present disclosure provides formulations for skin protection against radiation therapy. The present compositions provide effective entrapment of high water load, prolonged moisturizing effect of the upper skin layers and effective delivery of active agents (such as allantoin) to the skin as skin-protective remedy.

The present disclosure provides compositions and methods of use thereof in preventing skin damage. In some embodiments, the compositions and methods described herein are useful for prophylaxis of skin damages due to radiation or laser treatments. In some embodiments, the compositions and methods described herein are useful for prophylaxis of skin cancer.

The present compositions can be used for preventing damage to the skin including, but not limited to, burns and cancer, such as in subjects exposed to radiation. Without limiting the invention to a particular theory or mechanism of action, the compositions described herein act by allowing the penetration of radiation through the skin (e.g., the epidermis) to the targeted site rather than partially reflecting off the skin and causing damage.

In one embodiment, said composition is characterized by enhanced radiation skin permeability characteristics. In one embodiment, said composition is useful for preventing or reducing damage to skin exposed to radiation or laser. In one embodiment, said composition is useful for preventing occurrence or reoccurrence of skin cancer.

In one embodiment, there is provided a method of preventing or reducing skin associated disorders or damages in a subject in need thereof. The method may comprise topically applying to the subject an effective amount of the present composition. In one embodiment, said skin associated disorders or damages result from exposure to laser and/or radiation.

In one embodiment, the subject is undergoing radiotherapy. In one embodiment, the radiotherapy is external-beam radiation therapy. In one embodiment, the subject is undergoing laser treatment. In one embodiment, the skin associated disorders or damages are associated with overexposure to sun.

In one embodiment, the method comprises applying the composition to the skin of the subject prior to exposure to radiation and/or laser. In one embodiment, the composition is adsorbed to the skin of the subject prior to exposure to at least one of radiation or laser.

The composition may be topically applied to a subject prone to skin cancer reoccurrence. In one embodiment, reoccurrence of skin cancer does not reoccur over time. In some embodiments, the composition described herein is useful for preventing or reducing the occurrence of skin cancer, including, but not limited to, melanoma, basal cell carcinoma, squamous cell carcinoma, and Merkel cell carcinoma.

In additional embodiments, the composition described herein is useful for preventing skin damage which results for example from radiotherapy that is given to cancer patients. The composition described herein is useful for preventing skin damage due to laser, radiation or UV rays that are used for therapeutic and/or cosmetic purposes.

In some embodiments, without being limited to specific mechanisms, topically applying the compositions described herein results in enlargement of skin pores, thereby enhancing penetration of radiation through the skin to the targeted site rather than partially reflecting off the skin and causing damage.

In certain embodiments, topically applying the composition to the skin of a patient prior to radiation treatment reduces the radiation-induced damage to the skin of the patient. Oftentimes, due to skin damages induced by radiotherapy or laser treatments, the treatments are discontinued. In certain embodiments, the present composition and method allow treatment (e.g., radiotherapy or laser treatments) to continue over longer periods of time, thereby increasing the survival rates of cancer patients.

An adverse effect commonly seen in radiation or laser treatment (e.g., for treating cancer or for hair removal) is burns caused by partial absorption of heat or energy by the surrounding skin, which leads to skin damage including, but not limited to, burns, pigmentation and scars.

In some embodiments, the present composition is topically applied to the skin before the treatment or exposure to radiation or laser. In embodiments where the composition is applied for prevention of skin damage due to exposure to radiation or laser, the topical composition is applied at least or about 30 seconds, at least or about 1 minute, at least or about 5 minutes, at least or about 10 minutes, at least or about 15 minutes, at least or about 20 minutes, at least or about 30 minutes, at least or about 1 hour, or at least or about 2 hours, before the radiation therapy or laser treatment. In some embodiments, the composition may be applied at least or about 24 hours, at least or about 16 hours, at least or about 12 hours, at least or about 10 hours, at least or about 8 hours, at least or about 6 hours, at least or about 5 hours, at least or about 4 hours, or at least or about 3 hours, before the radiation therapy or laser treatment.

The present composition may be applied before exposure to radiotherapy. The composition may be applied during one to two weeks before radiotherapy, once to three times daily.

In some embodiments, the composition is applied to the area of the skin which is to be exposed to the radiation or laser. As used herein, the “area of the skin” exposed to radiation or laser includes skin surrounding the particular site of exposure, such as in a radius of at least or about 1 cm, at least or about 2 cm, at least or about 3 cm, at least or about 4 cm, at least or about 5 cm, at least or about 6 cm, at least or about 7 cm, at least or about 8 cm, at least or about 9 cm, at least or about 10 cm from the site of exposure.

In some embodiments, the topical composition described herein is applied to the skin of a human. In some embodiments, the topical composition described herein is applied to the skin of a mammal. In some embodiments, the topical composition described herein is applied to the skin of an animal. Non-limiting examples of subjects to be treated by the topical composition described herein include a human, horse, cow, camel, goat, sheep, dog, cat, non-human primate, mouse, rat, rabbit, hamster, guinea pig, and pig.

The terms “prevent” or “preventing” as used herein refers to prophylactic or preventative measures, wherein the object is to prevent or slow down (lessen) an undesired skin condition. The term “reducing” includes the administration of a compound of the present invention to prevent the onset of the symptoms, alleviating the symptoms, or eliminating the skin condition. By “reducing” is meant a negative alteration of at least or about 10%, 25%, 50%, 75%, or 100%.

As used herein, the term “skin damage” includes burns, ulcers, irritation, pain, itching fine and coarse wrinkles, pigmentation including mottled pigmentation, sallowness (i.e., yellow discoloration of the skin), freckles, as well as telangiectasias (i.e., dilation of small blood vessels under the skin) and elastosis (i.e., destruction of the elastic tissue in the skin). In some embodiments, said skin damage or disorder is radiation dermatitis or radiodermatitis.

### Radiotherapy (Radiation Therapy)

Radiation therapy works by directing ionizing radiation into the area being treated with the goal of damaging the genetic material of cancerous cells thereby making it impossible for these cells to divide. Accordingly, radiotherapy is an important tool in the fight against cancer and is used in the treatment many cancer patients. Other terms for radiotherapy include radiation therapy, x-ray therapy, electron beam therapy, cobalt therapy, or irradiation.

Radiotherapy is especially useful in cases where surgical removal of the cancer is not possible, where surgery might debilitate the patient, or where surgical debulking of the tumor has not absolutely removed all cancerous tissue. Radiotherapy is routinely used following surgery to destroy any cancer cells that were not removed by surgery. Further uses of radiotherapy are prior to surgery where it can “shrink” a previously inoperable tumor down to a manageable size to enable surgical excision.

Radiation therapy can also be used to help relieve symptoms of advanced cancer (such as bleeding or pain), even if a cure is not possible. Over one-third of the practice of radiation therapy is palliative. The typical intent of palliative treatment is to relieve pain quickly and maintain symptom control for the duration of the patient's life. Accordingly, treatment is usually tailored to the patient's clinical condition and overall prognosis. Palliative treatment is often complementary to analgesic drug therapies and may enhance their effectiveness because it can directly target the cause of pain.

Specifically, radiotherapy can be used to treat localized solid tumors, such as cancers of the skin, head and neck, brain, breast, prostate, cervix, and the like. Radiation therapy can also be used to treat cancers of the blood-forming cells and lymphatic system including leukemia and lymphoma respectively, and the like. Skin cells in the vicinity of the radiation or in the path of the radiation can be protected using the present invention.

External beam radiation therapy commonly uses photons to treat cancer. In some embodiments, radiation includes ultraviolet (UV) rays and particularly UV-A and UV-B radiation such as from the sun. Ultraviolet radiation between 290 nm and 320 nm (“UV-A”) has been known to rapidly produce damage to the human skin. Also, the human skin has been known to be affected by UV radiation between 320-400 nm (“UV-B”).

It is an object herein to ameliorate the negative effects of all radiotherapy regardless of the form of the photon or particle, including x-rays, gamma rays, UV rays including UV-A, UV-B and UV-C, neutrons, protons, and electrons including beta particles and the like. In some embodiments, laser includes laser beam used in laser therapy for cosmetic skin treatments and hair removal. Currently, 1064 nm wavelength is approved by FDA for permanent hair reduction.

X-rays are a very common form of radiation used in radiotherapy. Gamma rays are another form of photons used in radiotherapy. Gamma rays can be produced spontaneously as certain elements (such as radium, uranium, and cobalt 60), which release radiation as they decompose, or decay. Each element decays at a specific rate and can give off energy in the form of gamma rays and other particles. Typically x-rays and gamma rays have the same general effect on cancer cells.

External beam radiation therapy can be delivered by means of a linear accelerator. Typically, linear accelerators use powerful generators to create the high energy rays for external beam radiation therapy. Generally, linear accelerators can produce x-rays at various energies. The linear accelerator can include a special set of lead shutters, called collimators, which focus and direct the rays to the tumor. The linear accelerator can be a large “L-shaped” design which allows it to rotate and deliver radiation from all angles. Multiple angles allow the maximum amount of radiation to be delivered to the tumor while delivering a minimal amount of radiation to the surrounding healthy tissue. The formulations and methods described herein can be used in conjunction with collimators or other devices and methods that limit radiation exposure to normal cells.

The present compositions and methods described herein can ameliorate the effects of radiotherapy on skin cells. For example, the compositions and methods can ameliorate the effects of local-field radiation and wide-field radiation. Local field radiation relates to a narrow beam of radiation directed at the specific metastatic site or sites. Customarily, local field radiation has tended to be used for patients with a long life expectancy and fewer metastatic sites. In contrast, wide-field radiation employs a larger field of radiation and is often used to treat patients with a shorter life expectancy and multiple metastatic pain-causing sites.

Radiotherapy dosage is measured by the scientific unit rad (radiation absorbed dose) which is a radiation energy dose equal to energy of 100 ergs per gram of irradiated material. A patient who receives radiation therapy as a treatment for cancer can receive several thousand rads over a very short period of time (weeks or months). In contrast, a typical scanning x-ray contains far fewer rads. For example, modern mammography systems used to take x-ray images of the breast use approximately 0.1 to 0.2 rad dose per x-ray.

In general, radiation therapy is a local treatment. It typically affects the cells in the treated area. However, in addition to damaging cancer cells, radiation can also damage normal cells located in the treated area and particularly skin cells in the radiation path. Radiation side effects are typically restricted to the radiation portal and can be classified as acute, occurring during or immediately after the course of radiation therapy, or late, occurring months to years later. Acute radiation effects are more prominent with radiation schedules that deliver high total doses of radiation with small daily fractions; they generally begin at the end of the second week of therapy. Acute radiation effects, occurring at skin surfaces, usually consist of an inflammatory response such as skin erythema or pigmentation. Late radiation effects may arise without any preceding acute reactions. Fibrosis is the most common type oflate radiation injury and can be observed in many types of tissue, including skin.

Other skin conditions caused by radiation therapy include dry and moist desquamation. Dry desquamation, which is characterized by dry and flaky skin and pruritus in the area of irradiation. Moist desquamation, is characterized by sloughing of the epidermis, exposing the moist, raw, dermis layer of the skin.

One objective described herein is to ameliorate the negative effects of radiation therapy on normal skin cells, regardless of whether the effect is acute or late.

### Pharmaceutical Compositions

The present composition may contain a carrier. Non-limiting examples of carriers include, jojoba oil, coconut oil, aloe vera gel, cocoa butter, lecithin, almond oil, borage oil, canola oil, grape seed oil, olive oil, soybean oil, sunflower oil, wheat germ oil, apricot kernel oil, carrot oil, mango butter, evening primrose oil, black currant oil, avocado oil, macrocrystalline wax, paraffin, petrolatum, petroleum jelly, ozokerite, montan wax, beeswax, lanolin or a derivative, candelilla wax, ouricury wax, carnauba wax, Japan wax, sugarcane wax, cork fiber wax, shea oil, silicone oil, geranium oil and mixtures thereof. According to another embodiment, said carrier is selected from jojoba oil, coconut oil, aloe vera gel, and mixtures thereof.

In certain embodiments, the composition comprises a compound selected from the group consisting of vitamin E, vitamin A, benzoic acid, benzyl alcohol, cetyl alcohol, citric acid, glycerin, imidazolidinyl urea, isopropyl myristate, methylisothiazolinone, shea butter, sorbitan tristearate and combinations thereof.

If the present topical pharmaceutical compositions are formulated as an aerosol and applied to the skin as a spray-on, a propellant is added to a solution composition. A more complete disclosure of propellants useful herein can be found in Sagarin, Cosmetics Science and Technology, 2nd Edition, Vol. 2, pp. 443-465 (1972).

As used herein, “pharmaceutically acceptable” means that drugs, medicaments or inert ingredients which the term describes are suitable for use in contact with the tissues of humans and other animals without undue toxicity, incompatibility, instability, irritation, allergic response, and the like, commensurate with a reasonable benefit/risk ratio. For example, the term “pharmaceutically acceptable” can mean approved by a regulatory agency of the Federal or a state government or listed in the U. S. Pharmacopeia or other generally recognized pharmacopeia for use in animals, and more particularly in humans.

As used herein, “safe and effective amount” means an amount of compound or composition sufficient to significantly induce a positive modification in the condition to be treated, but low enough to avoid serious side effects (at a reasonable benefit/risk ratio), within the scope of sound medical judgment. The safe and effective amount of the compound or composition will vary with the particular condition being treated, the age and physical condition of the patient being treated, the severity of the condition, the duration of the treatment, the nature of concurrent therapy, the specific compound or composition employed, the particular pharmaceutically acceptable carrier utilized, and like factors within the knowledge and expertise of the attending physician.

The term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as jojoba oil, coconut oil, aloe-vera oil, peanut oil, soybean oil, mineral oil, sesame oil and the like, polyethylene glycols, glycerin, propylene glycol or other synthetic solvents.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; and agents for the adjustment of tonicity such as sodium chloride or dextrose may also envisioned.

In some embodiments, the pharmaceutical composition is water-based composition. As used herein the term “water-based” refers to a pharmaceutical composition whose primary solvent is water.

For topical application, a composition of the present invention or derivative thereof can be combined with a pharmaceutically acceptable carrier so that an effective dosage is delivered, based on the desired activity. The carrier can be in the form of, for example, and not by way of limitation, an ointment, cream, gel, paste, foam, aerosol, suppository, pad or gelled stick.

As used herein, “topical application” means directly laying on or spreading on outer skin.

The composition may further comprise one or more pharmaceutically acceptable excipients comprising lipids, oils, emulsifiers, initiators, pH adjusting agents, thickening agents, emollients, humectants, preservatives, antioxidants, and chelating agents.

The composition may further comprise one or more of the ingredients selected from: isopropyl myristate, aloe barbadensis leaf extract (e.g., juice), glyceryl stearate, cetyl alcohol, sweet almond oil, butyrospermum parkii, Propylene glycol, polyethylene glycol (PEG) 40 Stearate, jujuoba seed oil (Simmondsia chinensis), ethylhexyl methoxycinnamate, glycerin, sorbitan tristearate, tocopherol acetate (vitamin E acetate), butyl methoxydibenzoylmethane, germanium oil (or any other natural fragrance), imidazolidinyl urea, dehydroacetic acid, benzoic acid, sorbic acid and benzyl alcohol.

In some embodiments, the composition may include an emulsifying agent, or emulsifier. In embodiments, the emulsifier may be, for example, sodium lauryl sulfate, white waxes such as beeswax or paraffin wax, sesquioleates such as sorbitan sesquioleate or polyglyceryl-2-sesquioleate, ethoxylated esters of derivatives of natural oils such as the polyethoxylated ester of hydrogenated castor oil, silicone emulsifiers such as silicone polyols, anionic emulsifiers, fatty acid soaps such as potassium stearate and fatty acid sulphates like sodium cetostearyl sulphate, ethoxylated fatty alcohols, sorbitan esters, ethoxylated sorbitan esters, ethoxylated fatty acid esters such as ethoxylated stearates, ethoxylated mono, di-, and triglycerides, non-ionic self-emulsifying waxes, ethoxylated fatty acids, methylglucose esters such as polyglycerol-3 methyl glucose distearate, and combinations thereof. Various emulsions suitable for embodiments described herein and methods for preparing such emulsions are well known in the art and are described in, for example, Remington's Pharmaceutical Sciences, Mack Publishing Co., Easton, Pa., USA, which is hereby incorporated by reference in its entirety. In some embodiments, the formulation may include an emulsifier in an amount from about 1% to about 15%, and in other embodiments, the formulation may include from about 1% to about 10%, or from about 1% to about 5% emulsifier. If more than one emulsifier is used, the formulation may include from about 1% to about 5% or from about 1.5% to about 3% by weight of the formulation of each emulsifier.

In some embodiments, the compositions described herein may include one or more surfactants. Such embodiments are not limited by type of surfactant used; for example, in some embodiments, the one or more surfactants may be anionic surfactants such as alkyl sulfates, alkylether sulfates, alkylsulfonates, alkylaryl sulfonates, alkyl succinates, alkyl sulfosuccinates, N-alkoylsarcosinates, acyl taurates, acyl isethionates, alkyl phosphates, alkyl ether phosphates, alkyl ether carboxylates, .alpha.-olefinsulfonates, and the alkali metal and alkaline earth metal salts and ammonium and triethanolamine salts thereof. Such alkyl ether sulfates, alkyl ether phosphates and alkyl ether carboxylates can have between 1 and 10 ethylene oxide or propylene oxide units, and in some embodiments, 1 to 3 ethylene oxide units, per molecule. More specific examples include, but are not limited to, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium 15 dodecylbenzene sulfonate, triethanolamine dodecylbenzenesulfonate. In other embodiments, the one or more surfactants may be amphoteric surfactants such as, for example, alkylbetaines, alkylamidopropylbetaines, alkylsulfobetaines, alkylglycinates, alkylcarboxyglycinates, alkylamphoacetates or alpha-propionates, alkylamphodiacetates or alpha-dipropionates, and more specifically, cocodimethylsulfopropylbetaine, lauryl betaine, cocamidopropylbetaine or sodium cocamphopropionate.

In some embodiments, the composition may comprise emollients in an amount from about 8% to about 30% by weight of the formulation. In formulations that include more than one emollient, each emollient may be provided at about 0.05% to about 15% by weight of any one emollient. Emollients are well known in the art and are listed, for example, the International Cosmetic Ingredient Dictionary, Eighth Edition, 2000, which is hereby incorporated by reference in its entirety. In certain embodiments, the emollient may be fatty esters, fatty alcohols, or combinations thereof including, but not limited to, diisopropyl adipate, oleyl alcohol, lanolin, isopropyl myristate, isopropyl palmitate, caprylic/capric triglycerides, cetyl lactate, cetyl palmitate, hydrogenated castor oil, glyceryl esters, hydroxycetyl isostearate, hydroxy cetyl phosphate, isopropyl isostearate, isostearyl isostearate, diisopropyl sebacate, polyoxypropylene (5) poloxyethylene (20) cetyl ether (PPG-5-Ceteth-20), 2-ethylhexyl isononoate, 2-ethylhexyl stearate, C12 to C16 fatty alcohol, C12 to C16 fatty alcohol lactate, isopropyl lanolate, 2-ethyl-hexyl salicylate, and combinations thereof In some embodiments, the one or more emollients may be a combination of fatty alcohols. In certain embodiments, the one or more emollients may be 1-hexadecanol, acetylated lanolin, behenocyl dimethicone, C12-C15 alkyl benzoate, cetearyl octanoate, cocoglycerides, dicaprylate/dicaprate dimethi cone copolyol, dimethiconol, dioctyl adipate, glyceryl stearate, isocetyl alcohol, isohexadecane, isopentylcyclohexanone, isopropyl palmitate, lauryl lactate, mineral oil, methoxy peg-22/dodecyl glycol copolymer, myristyl lactate, ocryldodecyl neopentanoate, octyl cocoate, octyl palmitate, octyl stearate, octyldodecyl neopentanoate, polyglyceryl-4 isosterate, polyoxyl 40 stearate, polyoxymethylene urea, potassium sorbate, propylene glycol, propylene glycol isoceth-3 acetate, and propylene glycol myristyl ether acetate. In some embodiments, the emollient may be a high molecular weight saturated and unsaturated fatty alcohol such as, but not limited to, carbitol, lauryl alcohol, myristyl alcohol, cetyl alcohol, isocetyl alcohol, stearyl alcohol, isostearyl alcohol, hydroxystearyl alcohol, oleyl alcohol, ricinoleyl alcohol, behenyl alcohol, erucyl alcohol, 2-octyldodecanyl alcohol, cetearyl alcohol, lanolin alcohol, or the like. In particular embodiments, the emollient may be selected from cetyl alcohol, stearyl alcohol, lanolin oil, cod liver oil, or a combination thereof. In some embodiments, the formulation may comprise an emollient such as, without limitations, cetyl alcohol in an amount from about 2% to about 6%, stearyl alcohol in an amount from about 1% to about 3%, lanolin in an amount from about 5% to about 15%, cod liver oil in an amount from about 0.05% to about 5% or combinations thereof.

In some embodiments, the composition may include one or more viscosity modifiers. In some embodiments, the formulation may comprise from about 1% to about 10% or from about 1% to about 6% of each viscosity modifier. The viscosity modifier of such embodiments may generally include a high molecular weight compound such as, for example, carboxyvinyl polymer, carboxymethyl cellulose, polyvinyl pyrrolidone, hydroxyethyl cellulose, methyl cellulose, natural gum such as gelatin and tragacanth gum, and various alcohols such as polyvinyl alcohol. In other embodiments, the viscosity modifier may include ethanol or isopropyl alcohol. In some embodiments, the viscosity modifier may be a high molecular weight saturated and unsaturated fatty alcohol such as, but not limited to, carbitol, lauryl alcohol, myristyl alcohol, cetyl alcohol, isocetyl alcohol, stearyl alcohol, isostearyl alcohol, hydroxystearyl alcohol, oleyl alcohol, ricinoleyl alcohol, behenyl alcohol, erucyl alcohol, 2-octyldodecanyl alcohol, cetearyl alcohol, lanolin alcohol, and the like, and in certain embodiments, the viscosity modifier may be cetyl alcohol, stearyl alcohol or a combination thereof. In some embodiments, the formulation may comprise a viscosity modifier such as, without limitations, cetyl alcohol in an amount from about 2% to about 6%, stearyl alcohol in an amount from about 1% to about 3%, or combinations thereof.

Formulations of embodiments herein may further include a preservative. For example, preservatives useful in embodiments may include, but are not limited to, pentylene glycol, ethylene diamine tetra acetate (EDTA) and its salts, chlorhexidine and its diacetate, 5 dihydrochloride, digluconate derivatives, 1,1,1-trichloro-2-methyl-2-propanol, parachlorometaxylenol, polyhexamethylenebiguanide hydrochloride, dehydroacetic acid, diazolidinyl urea, 2,4-dichlorobenzyl alcohol, 4,4-dimethyl-1,3-oxazolidine, formaldehyde, glutaraldehyde, dimethylidantoin, imidazolidinyl urea, 5-chloro-2-methyl-4-iothiazolin-3-one, ortho-phenylphenol, benzyl alcohol, benzoic acid and its salts, 4-hydroxybenzoic acid and its methyl-, ethyl-, propyl-, isopropyl-, butyl-, isobutyl-esters (parabens), methylparaben, propylparaben, isopropylparabens, isobutylparabens, butylparabens, ethylparaben, trichlosan, 2-phenoxyethanol, phenyl mercuric acetate, quaternium-15, methylsalicylate, salicylic acid and its salts, sorbic acid and its salts, iodopropanyl butylcarbamate, calcium sorbate, zinc pyrithione, 5-bromo-Snitro-1,3-dioxane, 2-bromo-2-nitropropane-1,3-diol, sulfites, bisulfites, and benzalkonium chloride, phenoxyethanol, 2-phenoxyethanol, chloroxylenol, diazolidinyl urea, and combinations thereof. In certain embodiments, the formulation may include a combination of methylparaben and propylparaben. Preservatives may be provided in any concentration known in the art. In some embodiments, the composition may include preservatives in an amount from about 0.01% to about 3% by weight; and, in embodiments, the formulation may include from about 0.05% to about 1% or from about 0.05% to about 0.5% by weight of any one preservative.

The compositions of various embodiments may further include a chelating agent or combination of chelating agents. Examples of the chelating agents useful in various embodiments include, but are not limited to, alanine, sodium polyphosphate, sodium methaphosphate, citric acid, phosphoric acid, tartaric acid, ethylenediamine tetra acetic acid (Edetate, EDTA) and derivatives and salts thereof, dihydroxyethyl glycine, and combinations thereof. In particular embodiments, the chelating agent may be tetrasodium EDTA. The chelating agents may be provided in any effective amount. For example, in some embodiments, the formulation may include from about 0.01% to about 2% by weight chelating agent, and in other embodiments, the formulation may include from about 0.05% to about 0.5% or from about 0.05% to about 0.35% by weight chelating agent.

The formulations of certain embodiments may include one or more antioxidants. Numerous antioxidants are known in the art, and any such antioxidant may be used to prepare the formulations described herein. Examples of suitable antioxidants include, but are not limited to, amino acids such as glycine, histidine, tyrosine, trytophan and derivatives thereof, imidazoles such as urocanic acid and derivatives thereof, peptides, such as D,L-carnosine, D-carnosine, L-carnosine and derivatives thereof such as anserine, carotinoids, carotenes such as alpha.-carotone, beta.-carotene, lycopene, and derivatives thereof, chlorogenic acid and derivatives thereof, lipoic acid and derivatives thereof such as dihydrlipoic acid, aurothioglycose, propylthiouracil and other thiols such as thioredoxin, glutathione, cysteine, cystine, cystamine and glycosyl, N-acetyl, methyl, ethyl, propyl, amyl, butyl, lauryl, palmitoyl, oleyl, .alpha.-linoleyl, cholesteryl and glyceryl esters and salts thereof, dilauryl thiodipropionate, distearyl thiodipropionate, thiodipropionic acid and derivatives thereof such as esters, ethers, peptides, lipids, nucleotides, nucleosides, and salts, sulfoximine compounds such as buthionine sulfoximines, homocysteine sulfoximine, buthionine sulfones, penta-, hexa-, hepta-thionine sulfoximine, unsaturated fatty acids and derivatives thereof such as .alpha.-linolenic acid, linoleic acid, oleic acid, folic acid and derivatives thereof, ubiquinone and ubiquinol and derivatives thereof, vitamin C and derivatives thereof such as ascorbyl palmitate, magnesium ascorbyl phosphate, ascorbyl acetate, tocopherals and derivatives such as vitamin E acetate, vitamin A and derivatives such as vitamin A palmitate, vitamin B and derivatives thereof, coniferyl benzoate of benzoin resin, rutinic acid and derivatives thereof, alpha-glycosylrutin, ferulic acid, furfurylidene glucitol, carnosine, butyl hydroxytoluene, trihydroxy-butyrophenone, uric acid and derivatives thereof, mannose and derivatives thereof, superoxide dismutase, zinc and derivatives thereof such as ZnO, ZnSO4, selenium and derivatives thereof such as selenium methionine, stilbene and derivatives thereof such as stilbene oxide, trans-stilbene oxide and the like. In some embodiments, the antioxidants may include vitamin B, nordihydroguaiaretic acid, butylated hydroxyanisole (BHA), butylated hydroxytoluene (BHT), propyl gallate, erythorbate acid, sodium erythorbate, ascorbir palmitate, and ascorbir stearate, butyl hydroxyanisole, and gallic esters, and in particular embodiments, the one or more antioxidants may include BHT. The antioxidant may be provided in any suitable amount. For example in some embodiments, one or more antioxidants may be from about 0.001% to about 3% by weight of the formulation, and in other embodiments, the one or more antioxidants may be from about 0.01% to about 1% by weight of the formulation or from about 0.05% to about 1% by weight of the formulation.

In some embodiments, the formulation may include a solubilizing agent. In embodiments, the solubilizers may be, for example, hydrochloric acid, sodium hydroxide, glycine, cyclodextrin, liquid paraffin, hydrogenated castor oil, ethanol, glycerin, propylene glycol, dilute hydrochloric acid, hydrogenated oils, purified water, physiological saline, water for injection, Macrogol 4000, Polysorbate 80, or a combination thereof. In particular embodiments, the solubilizing agent may be propylene glycol, glycerin or a combination thereof. In embodiments, the solubilizing agent comprises from about 1% to about 20%, from about 1% to about 10% or from about 2% to about 8% by weight of the formulation.

In some embodiments, the composition may include one or more skin conditioners. Common skin conditioners include, for example, mineral oil, petrolatum, aliphatic alcohols, lanolin and its derivatives, fatty acids, glycol fatty acids, sugars, glycerin, propylene glycol, sorbitols, and polyethylene glycols, vitamins and herbal derivatives. Additional skin conditioners can be found in CTFA Cosmetic Ingredient Handbook, 1st Ed., 1988, which is hereby incorporated herein by reference in its entirety. In some embodiments, the one or more skin conditioners may include, but are not limited to, humectants, such as fructose, glucose, glycerin, propylene glycol, glycereth-26, mannitol and urea, pyrrolidone carboxylic acid, hydrolyzed lecithin, coco-betaine, cysteine hydrochloride, glutamine, polyoxypropylene, polyoxyethylene (PPG-15), sodium gluconate, potassium aspartate, oleyl betaine, thiamine hydrochloride, sodium laureth sulfate, sodium hyaluronate, hydrolyzed proteins, hydrolyzed keratin, amino acids, amine oxides, water-soluble derivatives of vitamins A, E and D, amino-functional silicones, ethoxylated glycerin, .alpha.-hydroxy acids and salts thereof, water-soluble fatty oil derivatives, such as PEG-24 hydrogenated lanolin, almond oil, grape seed oil and castor oil; numerous other water-soluble skin conditioners listed, and combinations thereof. In certain embodiments, the skin conditioners may include lanolin or lanolin derivatives, caprylic capric/triglyceride, diisopropyl adipate, and combinations thereof. Skin conditioners may be provided to various embodiments in any amount known in the art, and the amount of skin conditioner provided may vary depending upon the type of skin condition or combination of skin conditioners used. In general, the formulations of embodiments may include a conditioner in an amount from about 1% to about 30% by weight of the formulation or from about 1% to about 25% by weight of the formulation.

In embodiments, the formulation may further comprise a solvent. In some embodiments, the solvent may include one or more ingredients therein, with water being preferred in certain embodiments. Generally, the quantity of water used as a solvent may depend on the various other ingredients used. The solvent may be present in certain embodiments in a range of from about 10% to about 95% by weight, with certain embodiments including from about 40% to about 90%, from about 42% to about 87%, from about 42% to about 80%, from about 42% to about 75%, from about 42% to about 70%, or from about 42% to about 68% by weight of the formulation. The exact quantity of solvent may be dependent on the form of the product. For example, a product in lotion form may in certain preferred embodiments include more water than a product in spray form and a product in cream or butter form may include less water than a product in spray form. Deionized water is generally preferred. Other suitable solvent materials may also be used.

### Active Agents

The composition may comprise an active agent. The active agent may be water-soluble (e.g., allantoin), or lipophilic (e.g., essential oils dissolved in an alcohol such as ethanol). The active agent may be at a concentration ranging from about 0.01% to about 5%, from about 0.1% to about 1%, from about 1% to about 10%, about 5%, of the total weight of the composition. In one embodiment, the composition contains 4-5% essential oils and 15-16% ethanol of the total weight of the composition. In one embodiment, essential oils comprise lavender oil, eucalyptus oil and clove oil at a weight ratio of 1:1:1 dissolved in ethanol.

The present pharmaceutical compositions may contain 0.01 wt %-50 wt %, 0.05 wt %-50 wt % of the active agent(s), 0.1 wt %-25 wt %, 0.1 wt %-5 wt %, or 0.1 wt %-1 wt %. In any event, the composition may contain a quantity of active components in an amount effective to reduce or prevent skin conditions as described herein. In some embodiments, the pharmaceutical compositions may contain 0.01 wt %-50 wt %, 1 wt %-3 wt %, 0.1 wt %-10 wt % or 0.1 wt %-5 wt % allantoin. In some embodiments, the pharmaceutical compositions may contain 0.01 wt %-3 wt %, 1 wt %-3 wt %, 0.1 wt %-3 wt % or 0.1 wt %-1 wt % allantoin.

The active agents include, but are not limited to, antioxidants, cytotoxic agents, anti-inflammatory agents, antibiotics, antiviral agents, antifungal agents, immunosuppressants, immunomodulators, antibodies, antibodies-drug-conjugates, peptide-drug-conjugates, enzymes inhibitors, retinoids, vitamins (e.g., Vitamin A), and combinations thereof.

According to another embodiment, the active agent is uric acid or a derivative thereof. According to another embodiment, the derivative of uric acid is allantoin. Encompassed within this disclosure is all forms of allantoin, or a salt thereof, including, but not limited to, crystals, polymorphs, clathrates, solvates, hydrates, amorphous forms, co-crystals, and anhydrous forms. Embodiments of the present disclosure also relate to the salts of allantoin. The acids which are used to prepare the salts of the aforementioned compound are those which form non-toxic salts, i.e., salts containing pharmacologically acceptable anions, such as the hydrochloride, acetate, trifluoroacetic acid, tosylate, picrate, hydrobromide, hydroiodide, nitrate, sulfate, bisulfate, phosphate, acid phosphate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate salts.

### Process of Preparing the Compositions

Also encompassed by the present disclosure is a process for preparing a topical composition. The process may comprise:
(a) heating lipids and oil-soluble ingredients at a temperature ranging from about 65° C. to about 85° C. (e.g., about 75° C. or 80° C.) to obtain an oil phase;
(b) heating water-soluble ingredients in water at a temperature ranging from about 65° C. to about 85° C. (e.g., about 75° C. or 80° C.) to obtain an aqueous phase;
(c) emulsifying (e.g., by homogenization) the oil phase in the aqueous phase for a period of time (e.g., from about 20 minutes to about 2 hours, from about 30 minutes to about 1.5 hours, from about 30 minutes to about 1 hour, from about 30 minutes to about 50 minutes, or about 45 minutes) to obtain a liquid emulsion;
(d) cooling the liquid emulsion to a temperature ranging from about 25° C. to about 50° C. to obtain a semi-solid emulsion (e.g., under continuous agitation);
(e) adding a solution comprising at least one trace element to the semi-solid emulsion at a temperature ranging from about 30° C. to about 50° C. (e.g., about 40° C.) to obtain a mixture;
(f) adjusting a pH of the mixture to about 4-6 (or about 5-5.5), if the mixture's pH is not 4-6 (or about 5-5.5), to obtain the topical composition.

The process may further comprise step (g) of mixing a solution comprising at least one preservative and at least one antioxidant to the semi-solid emulsion after step (d) at a temperature ranging from about 30° C. to about 70° C. (e.g., about 50° C.). The at least one preservative may comprise imidazolidinyl urea, methylparaben, propylparaben, 1,3-Dimethylol-5,5-dimethyl hydantoin (DMDMH), butylated hydroxytoluene (BHT), or combinations thereof.

The oil phase may comprise beeswax, cetearyl alcohol, cetyl alcohol, glyceryl stearate, isopropyl myristate, paraffin oil, mineral oil, sesame oil, shea butter, sorbitan tristearate, a denatured alcohol (e.g., Alcohol SD 40), a polyethylene glycol (PEG) ester of stearic acid (e.g., PEG 100 stearate), or combinations thereof.

The aqueous phase may comprise glycerin, dimethicone, allantoin, cetearyl alcohol, PEG-20 stearate (e.g., Sabowax FL-20 is a blend of cetearyl alcohol and PEG-20 stearate), Sabowax Fl-20, polysorbate 20, polysorbate 80, or combinations thereof.

In step (e), the solution comprising at least one trace element may have a pH of about 3-4 or about 3-3.5.

Any components or combination of components known and useful in the art may be used to achieve an appropriate pH such as, for example, pH regulators including, but not limited to, lactic acid, citric acid, sodium citrate, glycolic acid, succinic acid, phosphoric acid, monosodium phosphate, disodium phosphate, oxalic acid, dl-malic acid, calcium carbonate, sodium hydroxide and sodium carbonate, sodium hydrogen carbonate, and ammonium hydrogen carbonate. In particular embodiments, the formulation may include, for example, citric acid or lactic acid as a pH modifier. In embodiments, the pH modifier may comprise from about 0.01% to about 1%, from about 0.05% to about 0.5%, from about 0.06% to about 0.15%, from about 0.06% to about 0.11%, or from about 0.06% to about 0.1% by weight of the formulation. In some embodiments, a pH adaptor may be added to the composition in order to maintain a specific pH range. The pH adaptor can be any material capable of adjusting PH values, where the types and molecular weight of the pH adaptor are without particular limitation.

### Kits

The present disclosure also provides for a kit comprising the present composition (in solid, semi-solid or liquid form, e.g., in the form of a cream, an ointment, a gel, lotion, liniment, paste or an emulsion). Such kits may include one or more containers comprising the present composition.

In some embodiments, the kit can comprise instructions for use in any of the methods described herein. In one embodiment, the kit comprises instructions for use the present composition before, during and/or after radiotherapy. The kit may further comprise a description of selecting a subject suitable for treatment based on identifying whether the subject is in need of the treatment. In some embodiments, the instructions comprise a description of administering the present composition to a subject who is in need of the treatment. In certain embodiments, instructions supplied in the kits are written instructions on a label or package insert.

Parts of a kit may be used simultaneously or chronologically staggered, i.e., at different points in time and with equal or different time intervals for any component of a kit. Time intervals can be selected to obtain the desired effect.

The kits provided herein are in suitable packaging. Suitable packaging includes, but is not limited to, a vial (e.g., a cryovial), a bottle, an ampoule, a tube (e.g., a cryotube), a bag, a flask, a jar, flexible packaging, and the like.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

Skincare and pharmaceutical remedies may be mixtures of substances that are miscible either in water or in oil. These mixtures may have at least two immiscible phases such as water and oil. A mixture may contain a dispersed phase in a continuous phase. The dispersed phase may be droplets, vesicles, or particles. An example of such a mixture is an emulsion. There are two main types of emulsions: oil-in-water (O/W) emulsions which are composed of small droplets of oil dispersed in a continuous water phase, and water-in-oil (W/O) emulsions which are composed of small droplets of water dispersed in a continuous oil phase. Oil-in-water emulsions are comfortable to use topically and cosmetically acceptable, as they are not very greasy and can be easily washed off by water. Although oil-in-water emulsions can rapidly deliver water to the skin, their moisturizing effect may only last for a relatively short period of time and consequently provide limited water retention for the skin. On the other hand, water-in-oil emulsions can be moisturizing, as they provide an oily barrier which reduces water loss from the stratum corneum.

In one embodiment, creams are semi-solid emulsions which may not as thermodynamically stable as the liquid emulsions. The inherent thermodynamic entropies of each of the emulsified phases (oils and water) dominate the separation of dispersed phases within time. The most common method to increase the physical stability of emulsions and to extend their shelf-life is to mix the two immiscible liquids in the presence of emulsifiers. The most common emulsifiers used in skincare and pharmaceutical topical products are known as surface active agents or surfactants.

Surfactants may be amphiphilic. They typically have a polar or hydrophilic (i.e., water-soluble) head and a non-polar (i.e., hydrophobic or lipophilic) tail. The measure of the surfactant capability to emulsify various proportions of oils and water is known as the Hydrophilic-Lipophilic-Balance (HLB). The degree of HLB enables the prediction of the surfactant solubility in either oil or water. Hence, the HLB classification scale provides the basis for selection of effective surfactants for the emulsification of oil-in-water or water-in-oil emulsions. Accordingly, surfactants with HLB values of less than 10 are considered as lipid-soluble (water-insoluble) and those that have HLB above 10 are water-soluble (lipid-insoluble). The HLB classification scale is used for selections of surfactants according to the required HLB of the emulsion constituents. Accordingly, most oil-in-water emulsions have an HLB range of 8-16, and water-in-oil emulsions usually have an HLB range of 3-6. In the common practice of emulsion preparations, the emulsification efficacy may be controlled by factors including the types and percentages of the selected surfactant. In some cases, stable emulsions may be obtained by a combination of two or more surfactants in the system.

The surfactants can act by their ability to adsorb to the surface of the dispersed droplets. Their typical structure and thermodynamics can enable their diffusion and orientation at the interface between the dispersed droplets and the continuous phase. This distinctive orientation can enable surfactants to reduce the interfacial tension between the dispersed phases and to reduce the surface energy and the thermodynamic instability of the dispersion system.

Surfactants can be classified according to the types of their polar head groups. Ionic surfactants can include carriers of net positive charge such as cationic surfactants, or negative charge such as anionic surfactants. An amphoteric surfactant can contain a head with two oppositely charged groups.

The emulsification mechanism of ionic surfactants can be determined by the type of charge of the head groups. Cationic and anionic surfactants can enable stable dispersion by increasing the electrostatic repulsion forces between the dispersed droplets. The dispersion system can be stabilized due to the electrostatic repulsions elicited by the ionic surfactants adsorbed at the interface of the dispersed droplets which diffuse the droplets and maintain them in the dispersed state.

In certain embodiments, the present composition is an oil-in-water emulsion containing at least one nonionic surfactant. The concentration of the nonionic surfactant(s) may range from about 1% to about 5% (w/w) in the composition. The nonionic surfactant may have an HLB ranging from about 8 to about 16.

Without limiting the invention to a particular theory or mechanism of action, the trace elements may act by inducing coacervation in emulsions made of nonionic surfactants. Nonionic surfactants may form a molecular barrier between the dispersed droplets known as the steric effect which is characterized by relatively weak intermolecular interactions. Therefore, the stability of nonionic surfactants-based emulsions is much more sensitive to the presence of ions in the system than the ionic surfactants-based emulsions.

Coacervates are organic-rich droplets formed via liquid-liquid phase-separation, mainly resulting from association of oppositely charged molecules. More specifically, coacervation refers to the production of coacervate colloidal droplets. When coacervation occurs, two liquid phases will co-exist: a dense, polymer-rich phase (coacervate phase or coacervate droplets) and a very dilute, polymer-deficient phase (dilute phase). In some cases, the coacervation approach is applied in order to increase the viscosity and consistency of the emulsion. This coacervation may be performed under controlled conditions, such as the temperature, the concentrations of the coacervation-inducing agents, and the type and concentration of the emulsifying surfactants. Coacervation-inducing agents may be substances that have ionic charges such as ionic polymers or salts (electrolytes). When salts are added to the emulsion, they reduce the existing repulsive forces (induced attraction) between the dispersive droplets. This effect may be limited to the specific and controlled conditions of the system. More specifically, the coacervation may be mostly dependent on the type of ions derived from the trace elements (anion or cation, monovalent or multivalent) and a range of concentrations of the conservation-inducing agent and the emulsion constituents. For example, an increase of the concentration of the coacervation-inducing agent may cause a loss of balance between the surfactants and the dispersed droplets and a disruption of the emulsion consistency and stability. Due to the electrostatic nature of the coacervation phenomenon, this mechanism can be relevant to salts and ionic surfactants-based emulsions rather than nonionic surfactants-based emulsions. In certain embodiments, specific trace elements may induce a controlled coacervation in a nonionic surfactant-based emulsion. More specifically, the specific trace elements may be cationic ions having at least bivalent charges, including, but not limited to, selenium (Se) and zinc (Zn). Surprisingly, these elements may elicit a transition of the dispersed unilamellar lipid-based vesicles to multilamellar structures in a nonionic surfactant-based oil-in-water emulsion.

Controlled coacervation may relate to a quantitative measurement of the ions charge and their concentration as a function of the electrostatic effects applied to the dispersion system. The measure of this effect may include the zeta (Z) potential. The Z-potential enables the detection of electrostatic fields between the dispersed droplets and it serves as a descriptive 30 measure to confirm the coacervation mechanism, the trace elements effects and its control. In the practice of emulsions, the Z-potential values of above −20 mV or +20 mV are considered as stable dispersion.

The present disclosure provides for the method of controlled coacervation and its physical effects of the cream constituents. More specifically, the present disclosure provides for the specific trace elements, their concentrations and conditions of use to control the coacervation of the oil-in-water cream base. The trace elements can affect the unique microscopic texture of the cream base and the morphology and physical properties of the dispersed lipid-based vesicles. The present method may produce a unique lamellar texture of the cream matrix. The lamellar matrix may comprise dispersed, multilamellar lipid-based vesicles that have distinctive morphology and function. The differentiated morphology and function of the dispersed multilamellar lipid-based vesicles can enhance the entrapment of water payload, to improve the encapsulation efficiency and the delivery of the active agent and to maximize the skin protection effect against radiation-induced skin damage for a prolonged period of time.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the examples of the present invention. At the very least, and not as an attempt to limit the application of the doctrine of equivalents to the scope of the claim, each numerical parameter should at least be construed in light of the number of reported significant digits and by applying ordinary rounding techniques. It should be noted that when “about” is at the beginning of a numerical list, “about” modifies each number of the numerical list. Further, in some numerical listings of ranges some lower limits listed may be greater than some upper limits listed. One skilled in the art will recognize that the selected subset will require the selection of an upper limit in excess of the selected lower limit. The term “about” in reference to a numeric value refers to ±10% of the stated numeric value. In other words, the numeric value can be in a range of 90% of the stated value to 110% of the stated value.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

As used herein, the percentage “% (w/w)” or “wt %” is percent weight to weight; the percentage “% (w/v)” is percent weight to volume (w in gram and v in milliliter); the percentage “% (v/v)” is percent volume to volume.

The term “substantially free” of an agent should be understood as meaning free of the agent, or that any amount of the agent present in the composition is so low so as not to have any effect on the function of the composition, on the outcome of the skin protection process or on the properties of the composition (for example the skin protection function) after it is taken out of the composition. In certain embodiment, the term “substantially free” of an agent means that the agent is less than or about 5% w/w (or % w/v, or % v/v), less than or about 4% w/w (or % w/v, or % v/v), less than or about 3% w/w (or % w/v, or % v/v), less than or about 2% w/w (or % w/v, or % v/v), less than or about 1% w/w (or % w/v, or % v/v), less than or about 0.5% w/w (or % w/v, or % v/v), less than or about 0.2% w/w (or % w/v, or % v/v), less than or about 0.1% w/w (or % w/v, or % v/v), less than or about 0.05% w/w (or % w/v, or % v/v), less than or about 0.02% w/w (or % w/v, or % v/v), or less than or about 0.01% w/w (or % w/v, or % v/v).

Additional objects, advantages, and novel features of the present invention will become apparent to one ordinarily skilled in the art upon examination of the following examples, which are not intended to be limiting. Additionally, each of the various embodiments and aspects of the present invention as delineated herein above and as claimed in the claims section below finds experimental support in the following examples.

### EXAMPLES

### Example 1 (Comparative Example)

According to a first aspect, the composition is an oil-in-water emulsion. The composition may comprise about 20% oil phase and about 70% aqueous phase with about 10% nonionic surfactants as emulsifiers. In one embodiment, the aqueous phase of the previous composition comprises 58% water, 0.5% of allantoin as active ingredient, 7% aloe vera gel, 4% of humectants and preservatives. The minerals extraction comprises 1% of a primary mineral-based blend of trace minerals and Jojoba wax homogenate at a weight ratio of 1:1. The primary mineral-based blend was mixed in the heated oil phase of the cream carrier below 80° C. A formulation may be produced as follows. Briefly, a primary blend of minerals and wax is firstly prepared by homogenization. This blend is then mixed in the heated oil phase of the cream carrier under 80° C. Specifically,
1. The minerals are extracted from fountain water as a dry powder mixture. The minerals include, but are not limited to: calcium (Ca) (e.g., about 50 mg/l), magnesium (Mg) (e.g., about 20 mg/l), potassium (K) (e.g., about 1 mg/l), sulfate (SO₄) (e.g., about 5 mg/l), silica (SiO₂) (e.g., about 10 mg/l), fluoride (F) (e.g., about 0.2 mg/l), bicarbonate (HCO₃) (e.g., about 250 mg/l), sodium (Na) (e.g., about 10 mg/l), chloride (Cl) (e.g., about 20 mg/l), and mixtures thereof.
2. Minerals selenium (Se) and zinc (Zn) are mixed at a 100:1 weight ratio. The minerals selenium (Se) and zinc (Zn) are then mixed with the mineral extract from Step 1 at a 1:1 weight ratio to form a mineral mixture. The final concentration of the total minerals in the composition is 0.5% w/v.
3. The mineral mixture is blended with jojoba wax at a 1:1 weight ratio by homogenization at a temperature under 10° C. to form a semi-solid mineral-wax blend.
4. The semi-solid mineral-wax blend is mixed in the oil phase of the cream carrier under 80° C.
5. The composition contains 58 wt % water, 20 wt % oils, 9 wt % nonionic surfactants, 0.5 wt % allantoin as active ingredient, 7 wt % aloe vera gel, 4 wt % of humectants and preservatives.
6. The oil phase is emulsified in the water phase by homogenization.
7. The emulsion is cooled down to 50° C.
8. The preservatives are added to the cream base.
9. The composition's pH is adjusted to 5.5 using citric acid.

### Example 2

In certain embodiments, the present composition may be the formulation in Tables 1a-1j. The formulations may have an HLB of 10-13, pH 5-5.5, and Zeta potential ranging from −20 mV to −40 mV.

TABLE 1a

| | Percentage |
|---|---|
| Compound | (% w/w) |
| Water (e.g., doubled distilled water or DDW) | 45.32 |
| Glycerin | 5.50 |
| Tween 20 | 1.83 |
| Allantoin | 0.50 |
| Polysorbate 80 (e.g., Tween 80) | 1.83 |
| Cetyl Alcohol C16 | 2.75 |
| Isopropyl Myristate (IPM) | 4.59 |
| Silicone 350 | 0.46 |
| Paraffin Oil | 5.50 |
| Span 60 | 1.83 |
| Shea Butter | 2.75 |
| Glyceryl stearate SE | 1.38 |
| Beeswax | 3.67 |
| butylated hydroxy toluene (BHT) | 0.18 |
| Sabo wax fl 20 | 5.50 |
| Sabowax fl 65K | 1.83 |
| Sesame Oil | 1.83 |
| Water (e.g., DDW, to dissolve the preservative(s)) | 0.73 |
| 1,3-Dimethylol-5,5-dimethyl hydantoin (DMDMH) | 0.55 |
| BIOPUR 100 | 0.37 |
| Selenium and Zinc (weight ratio 100:1) | 0.02 |
| Water (e.g., DDW, to dissolve Se/Zn) | 10.00 |
| Methylparaben | 0.28 |
| Propylparaben | 0.08 |
| Alcohol | 0.74 |
| Triethanolamine (TEA) | 0.01 |

TABLE 1b

| | Percentage |
|---|---|
| Compound | (% w/w) |
| DDW | 46.00 |
| Glycerin | 5.50 |
| Tween 60 | 3.60 |
| Allantoin | 0.50 |
| Cetearyl Alcohol | 1.50 |
| Cetyl Alcohol C16 | 3.00 |
| IPM Isopropyl Myristate | 4.00 |
| Silicone 350 | 0.50 |
| Mineral Oil | 5.50 |
| Span 60 | 1.80 |
| Cocoa Butter | 3.00 |
| Sodium Stearate | 0.08 |
| Beeswax | 4.00 |
| Glyceryl monostearate | 1.00 |
| PEG-20 Stearate | 4.00 |
| PEG-100 Stearate | 1.00 |
| Almond Oil | 2.00 |
| Selenium and Zinc | 0.02 |
| (weight ratio 100:1) | |
| Selenium and Zinc (100:1) in DDW | 10.00 |
| Preservatives | 1.50 |
| Alcohol SD40 | 0.74 |
| TEA | 0.01 |
| Water (e.g., DDW, to dissolve Se/Zn) | 0.73 |

TABLE 1c

| | Percentage |
|---|---|
| Compound | (% w/w) |
| Water (e.g., doubled distilled water or DDW) | 45.62 |
| Glycerin | 5.5 |
| Diclofenac sodium | 0.2 |
| Cetyl Alcohol C16 | 2.75 |
| Span 80 | 1.24 |
| Tween 80 | 4.25 |
| Isopropyl Myristate (IPM) | 4.59 |
| Silicone 350 | 0.46 |
| Paraffin Oil | 5.5 |
| Shea Butter | 2.75 |
| Glyceryl stearate SE | 1.38 |
| Beeswax | 3.67 |
| Sesame Oil | 1.83 |
| Sab wax fl 20 | 5.5 |
| Sab wax fl 65K | 1.83 |
| Water (e.g., DDW, to dissolve the preservative(s)) | 0.73 |
| Selenium and Zinc (weight ratio 100:1) | 0.02 |
| Water (e.g., DDW, to dissolve Se/Zn) | 10 |
| Preservatives | 1.46 |
| Alcohol | 0.74 |
| Triethanolamine (TEA) | 0.01 |

TABLE 1d

| | Percentage |
|---|---|
| Compound | (% w/w) |
| DDW | 46.00 |
| Glycerin | 5.50 |
| Tween 80 | 4.25 |
| Allantoin | 0.50 |
| Cetearyl Alcohol | 1.50 |
| Cetyl Alcohol C16 | 3.00 |
| IPM Isopropyl Myristate | 4.00 |
| Silicone 350 | 0.50 |
| Mineral Oil | 5.50 |
| Span 80 | 1.24 |
| Cocoa Butter | 3.00 |
| Sodium Stearate | 0.08 |
| Beeswax | 4.00 |
| Glyceryl monostearate | 1.00 |
| PEG-20 Stearate | 4.00 |
| PEG-100 Stearate | 1.00 |
| Almond Oil | 2.00 |
| Selenium and Zinc | 0.02 |
| (weight ratio 100:1) | |
| Selenium and Zinc (100:1) in DDW | 10.00 |
| Preservatives | 1.50 |
| Alcohol SD40 | 0.74 |
| TEA | 0.01 |
| Water (e.g., DDW, to dissolve Se/Zn) | 0.73 |

TABLE 1e

| | Percentage |
|---|---|
| Compound | (% w/w) |
| Water (e.g., doubled distilled water or DDW) | 45.62 |
| Glycerin | 5.5 |
| Diclofenac sodium | 0.2 |
| Cetyl Alcohol C16 | 2.7 |
| Span 60 | 1.8 |
| Tween 60 | 3.6 |
| Isopropyl Myristate (IPM) | 4.5 |
| Silicone 350 | 0.46 |
| Paraffin Oil | 5.5 |
| Shea Butter | 2.75 |
| Glyceryl stearate SE | 1.38 |
| Beeswax | 3.63 |
| Sesame Oil | 1.83 |
| Sab wax fl 20 | 5 |
| Sabwax fl 65K | 1.83 |
| Water (e.g., DDW, to dissolve the preservative(s)) | 0.73 |
| Selenium and Zinc (weight ratio 100:1) | 0.02 |
| Water (e.g., DDW, to dissolve Se/Zn) | 10 |
| Preservatives | 1.46 |
| Alcohol | 0.74 |
| Triethanolamine (TEA) | 0.01 |
| TEA | 0.01 |
| Water (e.g., DDW, to dissolve Se/Zn) | 0.73 |

TABLE 1f

| | Percentage |
|---|---|
| Compound | (% w/w) |
| DDW | 46.00 |
| Glycerin | 5.50 |
| Tween 60 | 2.35 |
| Allantoin | 0.50 |
| Cetearyl Alcohol | 1.50 |
| Cetyl Alcohol C16 | 3.00 |
| IPM Isopropyl Myristate | 4.00 |
| Silicone 350 | 0.50 |
| Mineral Oil | 5.50 |
| Cocoa Butter | 3.00 |
| Glyceryl stearate SE | 1.83 |
| Beeswax | 4.00 |
| PEG-20 Stearate | 4.00 |
| Arlacel 165 | 3.30 |
| Almond Oil | 2.00 |
| Selenium and Zinc | 0.02 |
| (weight ratio 100:1) | |
| Selenium and Zinc (100:1) in DDW | 10.00 |
| preservatives | 1.50 |
| Alcohol SD40 | 0.74 |
| TEA | 0.01 |
| Water (e.g., DDW, to dissolve Se/Zn) | 0.73 |

TABLE 1g

| | Percentage |
|---|---|
| Compound | (% w/w) |
| DDW | 46.00 |
| Glycerin | 5.50 |
| Tween 60 | 3.60 |
| Allantoin | 0.50 |
| Cetearyl Alcohol | 1.50 |
| Cetyl Alcohol C16 | 3.00 |
| IPM Isopropyl Myristate | 4.00 |
| Silicone 350 | 0.50 |
| Paraffin Oil | 5.50 |
| Span 60 | 1.80 |
| Shea Butter | 3.00 |
| Sodium Stearate | 0.08 |
| Beeswax | 4.00 |
| Glyceryl monostearate | 1.00 |
| PEG-20 Stearate | 4.00 |
| PEG-100 Stearate | 1.00 |
| Sesame Oil | 2.00 |
| Selenium and Zinc | 0.02 |
| (weight ratio 100:1) | |
| Selenium and Zinc (100:1) in DDW | 10.00 |
| Preservatives | 1.50 |
| Alcohol SD40 | 0.74 |
| TEA | 0.01 |
| Water (e.g., DDW, to dissolve Se/Zn) | 0.73 |

TABLE 1h

| | Percentage |
|---|---|
| Compound | (% w/w) |
| DDW | 46.00 |
| Glycerin | 5.50 |
| Tween 60 | 2.35 |
| Allantoin | 0.50 |
| Cetearyl Alcohol | 1.50 |
| Cetyl Alcohol C16 | 3.00 |
| IPM Isopropyl Myristate | 4.00 |
| Silicone 350 | 0.50 |
| Paraffin Oil | 5.50 |
| Shea Butter | 3.00 |
| Glyceryl stearate SE | 1.83 |
| Beeswax | 4.00 |
| PEG-20 Stearate | 4.00 |
| Arlacel 165 | 3.30 |
| Carrot Oil | 2.00 |
| Selenium and Zinc | 0.02 |
| (weight ratio 100:1) | |
| Selenium and Zinc (100:1) in DDW | 10.00 |
| preservatives | 1.50 |
| Alcohol SD40 | 0.74 |
| TEA | 0.01 |
| Water (e.g., DDW, to dissolve Se/Zn) | 0.73 |

TABLE 1i

| | Percentage |
|---|---|
| Compound | (% w/w) |
| Water (e.g., doubled distilled water or DDW) | 45.62 |
| Glycerin | 5.5 |
| Diclofenac sodium | 0.2 |
| Cetyl Alcohol C16 | 2.7 |
| Span 60 | 1.8 |
| Tween 60 | 3.6 |
| Isopropyl Myristate (IPM) | 4.5 |
| Silicone 350 | 0.46 |
| Mineral Oil | 5.5 |
| Shea Butter | 2.75 |
| Glyceryl stearate SE | 1.38 |
| Beeswax | 3.63 |
| Castor Oil | 1.83 |
| Sab wax fl 20 | 5 |
| Sabwax fl 65K | 1.83 |
| Water (e.g., DDW, to dissolve the preservative(s)) | 0.73 |
| Selenium and Zinc (weight ratio 100:1) | 0.02 |
| Water (e.g., DDW, to dissolve Se/Zn) | 10 |
| Preservatives | 1.46 |
| Alcohol | 0.74 |
| Triethanolamine (TEA) | 0.01 |
| TEA | 0.01 |
| Water (e.g., DDW, to dissolve Se/Zn) | 0.73 |

TABLE 1j

| | Percentage |
|---|---|
| Compound | (% w/w) |
| DDW | 46.00 |
| Glycerin | 5.50 |
| Tween 80 | 4.25 |
| Allantoin | 0.50 |
| Cetearyl Alcohol | 1.50 |
| Cetyl Alcohol C16 | 3.00 |
| IPM Isopropyl Myristate | 4.00 |
| Silicone 350 | 0.50 |
| Paraffin Oil | 5.50 |
| Span 80 | 1.24 |
| Shea Butter | 3.00 |
| Sodium Stearate | 0.08 |
| Beeswax | 4.00 |
| Glyceryl monostearate | 1.00 |
| PEG-20 Stearate | 4.00 |
| PEG-100 Stearate | 1.00 |
| Almond Oil | 2.00 |
| Selenium and Zinc | 0.02 |
| (weight ratio 100:1) | |
| Selenium and Zinc (100:1) in DDW | 10.00 |
| Preservatives | 1.50 |
| Alcohol SD40 | 0.74 |
| TEA | 0.01 |
| Water (e.g., DDW, to dissolve Se/Zn) | 0.73 |

### Example 3

Known skincare compositions contain relatively low concentrations (e.g., 9 wt %) of surfactants. The method of production was based on complex processes, including, preparation of a primary blend of the minerals and wax, homogenization under controlled temperature of less than 10° C., followed by incorporation of the primary blend to the oil phase of the cream carrier under 80° C.

Compared to existing methods, a new method has been developed to be easily adjustable for mass production of compositions with relatively high viscosity which offer prolonged skin protection and do not require frequent applications. The new method improves the rheological properties of the compositions.

Surprisingly, we found that when the concentration of the surfactants increased (e.g., to about 15% or about 20%), what we termed as embryonic lipid-based vesicles formed. These lipid-based vesicles are stably dispersed in the aqueous phase and had a unique lamellar morphology.

In one embodiment, a solution containing trace elements (e.g., minerals) are added to the composition during the cooling stage. Another unexpected finding was that addition of the minerals to the surfactants-enriched formulation, during the cooling stage of the cream base, was compatible with, and did not disrupt, the dispersion uniformity and stability. Without wishing to be bound by any specific mechanism, the minerals may play a role in skin protection due to their physical effect on the cream base.

We studied the physical roles of the minerals in the cream base. To this end, we designed and tested a set of new cream carriers followed by evaluations of the rheological properties, z-potential measurements and microscopic morphology of the dispersed constituents as well as cross-section analysis of the cream matrix and textures.

We discovered a novel phenomenon of trace elements induced physical effects in nonionic surfactant-based emulsion. We were surprised to discover that our specific mixture and method of mixing of the trace elements, such as selenium (Se), and zinc (Zn), induced a coacervation in nonionic surfactant-based emulsion. These findings enabled us to discover and develop novel compositions and methods of production of controlled coacervation and transition of dispersed unilamellar lipid-based vesicles into stable colloidal dispersion of multilamellar vesicles in oil-in-water emulsions.

A method of production of the present composition (Table 1a) is as follows.
1. The water-soluble ingredients are dissolved in water (e.g., double distilled water).
2. The water phase is heated to about 80° C.
3. The oil-soluble ingredients are mixed in the oil phase.
4. The oil phase is heated to about 75° C.
5. The oil phase is then emulsified in the water phase by homogenization for about 45 minutes.
6. The emulsion is cooled down to about 50° C.
7. The preservatives are dissolved in water and added to the emulsion.
8. Selenium (Se) and zinc (Zn) are dissolved in water at a 0.2% w/w concentration to form a Se/Zn solution. The Se/Zn solution is then added to the emulsion at a weight ratio of 1:9 (the Se/Zn solution has a 10% w/w concentration of the mixture) at about 40° C. The final concentration of the selenium (Se) and zinc (Zn) in the composition is 0.02% w/w.
9. The pH of the composition is adjusted to 5.0 with TEA if necessary (if the pH is not 5.0).

The above method includes the procedure of emulsification and the coacervation induced by the trace elements selenium (Se) and zinc (Zn). Compared to the formulation in Example 1, the content of nonionic surfactants was increased from 9% (Example 1) to about 15% (Examples 2 and 3). In Example 1, the trace elements selenium (Se) and zinc (Zn), in addition to a plurality of other minerals, were first blended by homogenization with jojoba wax under temperature of <10° C. and then added, as a minerals-wax homogenate, to the heated/melted oil phase at 80° C. In Examples 2 and 3, only trace element selenium (Se) and zinc (Zn) were used and added to the cream base during the cooling stage at 40° C. in order to induce the controlled coacervation.

### Example 4

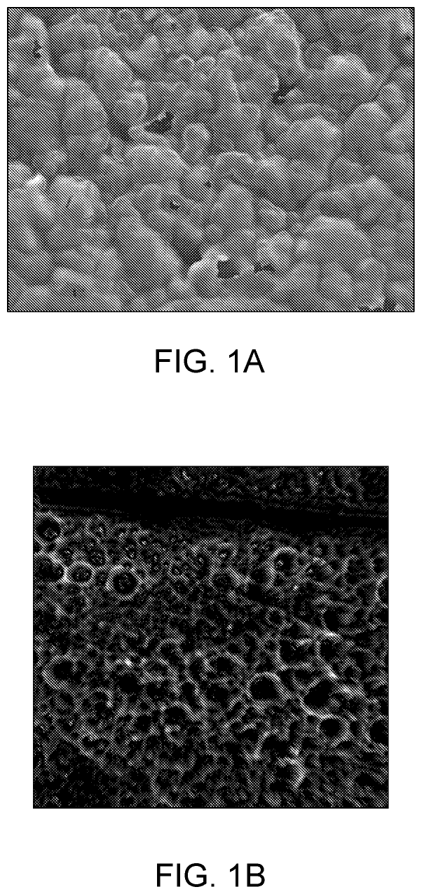

FIG. 1A shows the cryo-scanning electron microscopy (cryo-SEM) image of the dispersed lipid-based vesicles in an embodiment of the present composition.

The cryo-SEM image (FIG. 1A) shows the dispersed microparticles as embedded in the cream base of an embodiment of the present composition. The microparticles had a spherical morphology and a size range of about 1-5 μm and they were embedded in the cream matrix as clusters with an indicated size range of 5-10 μm. This image shows the upper view of the surface of the embedded microparticles and the upper view of the surface of the cream matrix.

FIG. 1B shows the confocal (bright field) image of the dispersed lipid-based vesicles in the composition.

The confocal image (FIG. 1B) shows the dispersed microcapsules of the Se/Zn coacervation system of an embodiment of the present composition. The white borders represent the lipid phase and the black color represent the water phase. As can be seen, the capsules contain significant encapsulated water payload.

### Example 5

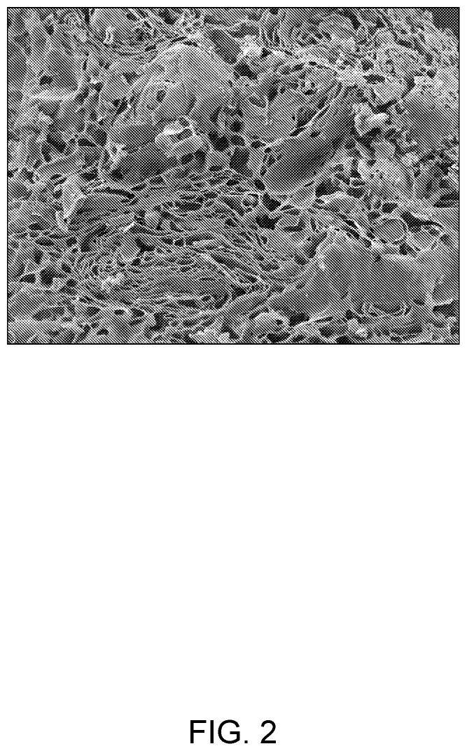

FIG. 2 is the cryo-SEM image showing the lamellar texture of the cream matrix of an embodiment of the present composition.

The cryo-SEM image (FIG. 2) shows the cross section of the cream matrix. The cream matrix has a lamellar texture characterized by dense multilamellar forms. This texture enables the entrapment of water in the cream matrix in addition to the encapsulated water in the vesicles.

### Example 6

The comparative morphology of the trace elements selenium (Se) and zinc (Zn) induces coacervation of the dispersed lamellar lipid-based vesicles of an embodiment of the present composition.

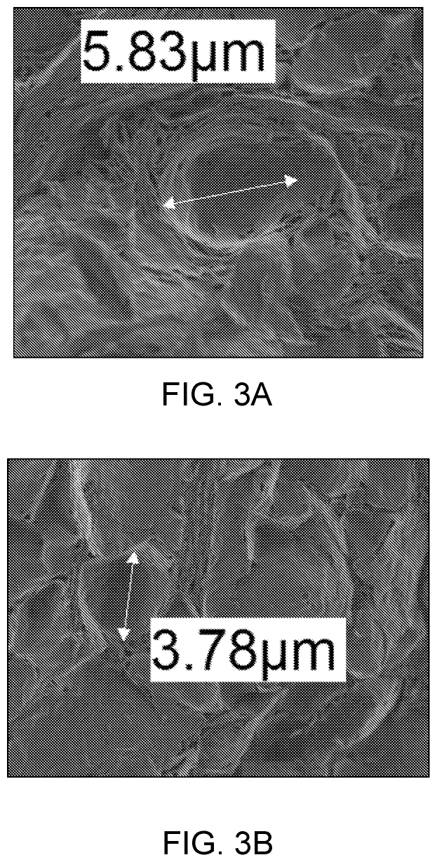

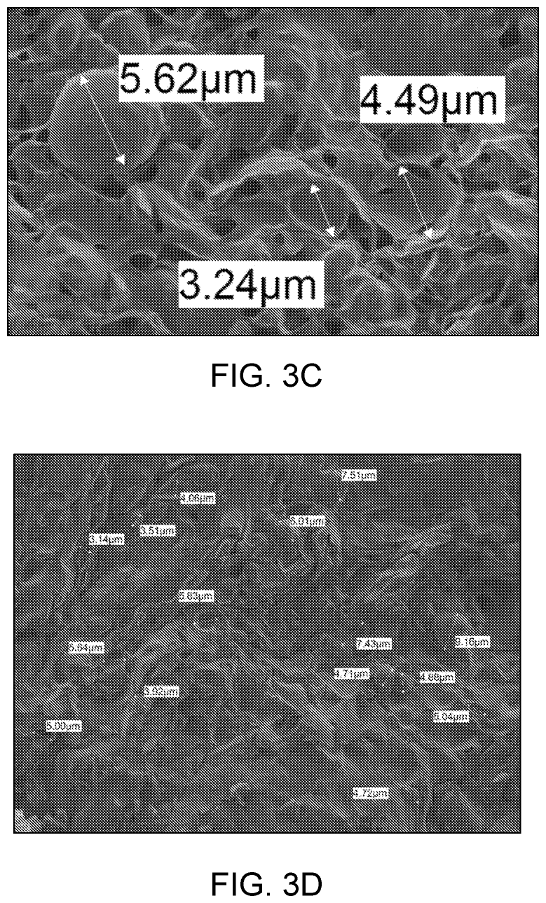

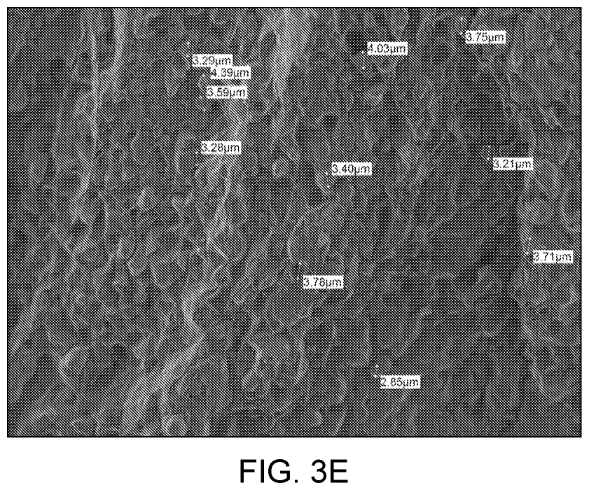

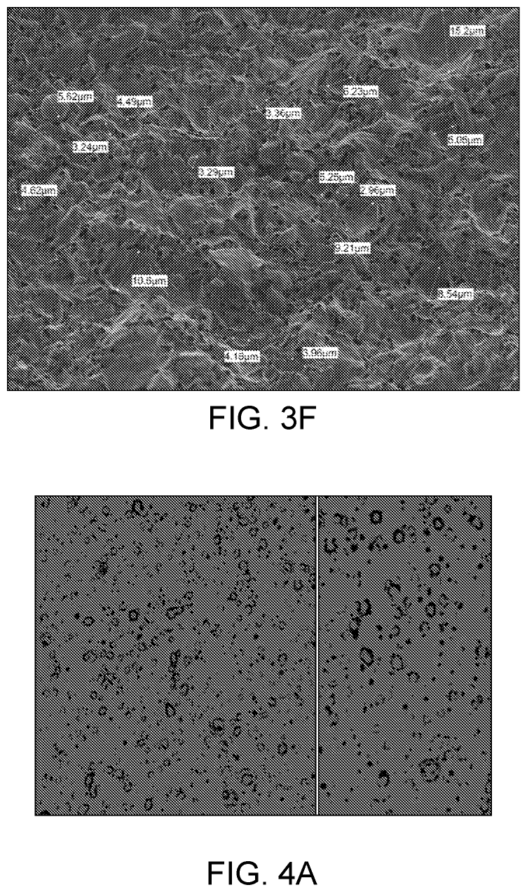

FIG. 3A is a cryo-SEM image showing the multilamellar lipid-based vesicle as an example of the coacervation induced by selenium (Se) and zinc (Zn).

The cryo-SEM image (FIG. 3A) shows a multilamellar lipid-based vesicle as an example of the coacervation induced by selenium (Se) and zinc (Zn) in an embodiment of the present composition produced by the method in Example 3. The core of the vesicle is surrounded by a very tight multilamellar structure oriented at the perimeter of the vesicle. The matrix that enclosed to the vesicle is also characterized with lamellar morphology. The core and the multilamellar structures of the vesicle enable the encapsulation of the water phase. The size of the multilamellar lipid-based vesicles is about 5-6 μm. These multilamellar vesicles (MLVs) have significant capacity for water entrapment. The composition has a relatively high viscosity as a result of the coacervation.

FIG. 3B is a cryo-SEM image showing the lamellar lipid-based vesicles in the absence of trace elements.

The cryo-SEM image (FIG. 3B) shows a unilamellar lipid-based vesicle in the absence of trace elements. The composition comprises a relatively high number of vesicles per area. These vesicles have distinctive morphology characterized by a smaller core and a relatively thin unilamellar surface layer. Their sizes are about 3-4 μm, smaller than the coacervated multilamellar vesicles depicted in FIG. 3A. The lack of trace elements results in less lamellar forms in the matrix. These small unilamellar vesicles (SUVs) have a smaller capacity for water entrapment. The cream has less viscosity than the coacervated formulation of FIG. 3A.

FIG. 3C is a cryo-SEM image showing an example of the lamellar lipid-based vesicles of the composition produced with a mineral extract but without selenium (Se) and zinc (Zn).

The cryo-SEM image (FIG. 3C) shows a unilamellar lipid-based vesicle produced with the mineral extract as described in Example 1 but without the trace elements selenium (Se) and zinc (Zn). The matrix comprises relatively high number of vesicles per area. The morphology is characterized by a high variability of vesicular size range of 3-11 μm. All observed vesicles are large unilamellar vesicles (LUVs). The matrix of the composition lacks lamellar structures and there are many pores observed in the matrix. This cream base has a lower water entrapment capacity and lower viscosity in comparison to the coacervated formulation of Example 6 which contained only selenium (Se) and zinc (Zn) as trace elements. This data confirmed that selenium (Se) and zinc (Zn) can play an important role in the coacervation of the present composition.

Table 2 shows the comparative microscopic evaluations of trace element selenium (Se) and zinc (Zn) on the coacervation of the compositions.

TABLE 2

| | Group 1 | Group 2 | Group 3 |
|---|---|---|---|
| Vesicle | Plus Se/Zn | No minerals | Minerals |
| Size | (about | (DDW) | extract |
| (diameter) | 0.02 wt %) | | without |
| (μm) | | | Se/Zn |
| | 7.51 | 3.75 | 15.2 |
| | 6.01 | 4.03 | 5.05 |
| | 4.06 | 3.29 | 6.23 |
| | 3.51 | 4.39 | 3.36 |
| | 3.14 | 3.59 | 2.96 |
| | 5.83 | 3.28 | 5.25 |
| | 7.4 | 3.4 | 3.29 |
| | 9.16 | 3.21 | 4.49 |
| | 4.85 | 3.71 | 5.62 |
| | 4.71 | 2.85 | 3.24 |
| | 5.04 | 3.78 | 4.62 |
| | 4.72 | | 2.96 |
| | 3.92 | | 9.21 |
| | 5 | | 8.54 |
| | 5.64 | | 3.96 |
| | | | 4.18 |
| | | | 10.6 |
| Mean | 5.4 | 3.6 | 5.8 |
| (μm) | | | |
| SD | 1.6 | 0.4 | 3.2 |
| (standard | | | |
| deviation) | | | |

TABLE 3

| | | Statistical |
|---|---|---|
| Analyzed Statistical | Probability | Significance |
| Group 1 vs Group 3 | 0.321235 | 67.9 |
| Group 1 vs Group 2 | 0.000835 | 99.9 |
| Group 2 vs Group 3 | 0.017899 | 98.2 |

The data shows the significant higher size of coacervated multilamellar vesicles (MLVs) of Group 1 with trace elements Se/Zn compared to Group 2 of the small unilamellar vesicles (SUVs) which had the same basic composition as Groups 1, 2 and 3 but without any minerals. Note the high variability observed in Group 3 of the LUVs with the plurality of minerals. FIGS. 3D-3F show the cryo-SEM images used for this comparative analysis.

FIG. 3D is a cryo-SEM image of an embodiment of present composition with the trace element selenium (Se) and zinc (Zn).

FIG. 3E is a cryo-SEM image of the composition with no minerals.

FIG. 3F is a cryo-SEM image of the composition with the mineral extracts but without Se/Zn.

### Example 7

Confocal microscopy was used to compare the morphologies of the trace elements selenium (Se) and zinc (Zn) induced coacervation of the dispersed lamellar lipid-based vesicles versus the effect of the minerals extract.

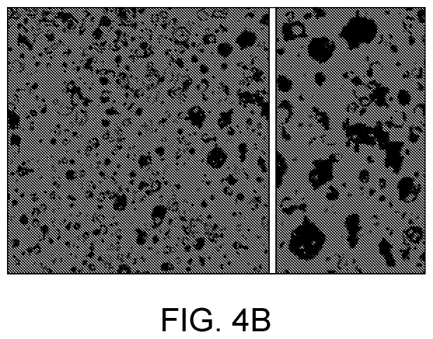

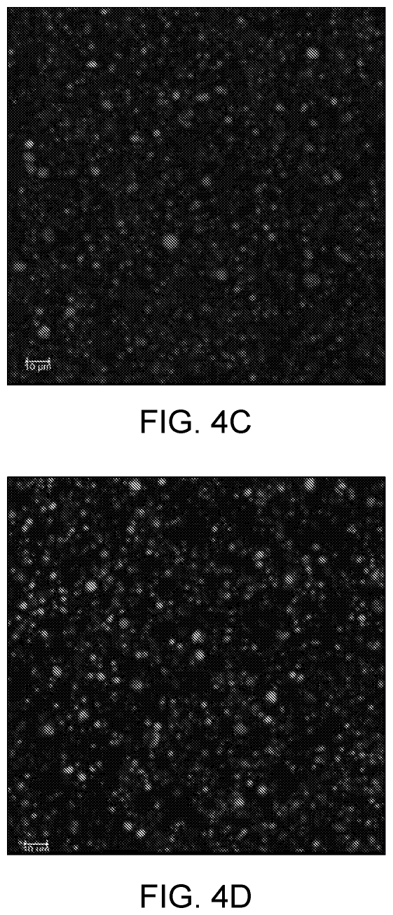

FIG. 4A is a confocal microscopy image showing the composition with the minerals extract but without Se/Zn.

The confocal bright field images (FIG. 4A) depict the dispersed lamellar vesicles with clear perimeter borders of the LUVs of the minerals in an embodiment of the present composition with Se/Zn.

FIG. 4B is a confocal microscopy image showing an embodiment of present composition with selenium (Se) and zinc (Zn).

The confocal bright field images (FIG. 4B) depict the dispersed multilamellar vesicles with significantly thicker borders of the MLVs in an embodiment of the present composition with Se/Zn versus the mineral extract formulation without the trace elements Se/Zn.

FIG. 4C is a confocal microscopy image (Nile-red) showing the composition with the mineral extract but without Se/Zn.

FIG. 4D is a confocal microscopy image (Nile-red) showing an embodiment of present composition with selenium (Se) and zinc (Zn).

FIGS. 4C and 4D are confocal images following the staining of samples with Nile Red, a selective fluorescent stain for lipids. The data show the significant increase of lipid staining in an embodiment of the present composition with Se/Zn compared to the formulation with the mineral extract (without Se/Zn). This data indicated an increased density of the lipids in the coacervated MLVs versus the LUVs counterparts.

### Example 8

We studied comparative z-potential measurements of the mineral extract versus the Se/Zn effect in an embodiment of the present composition.

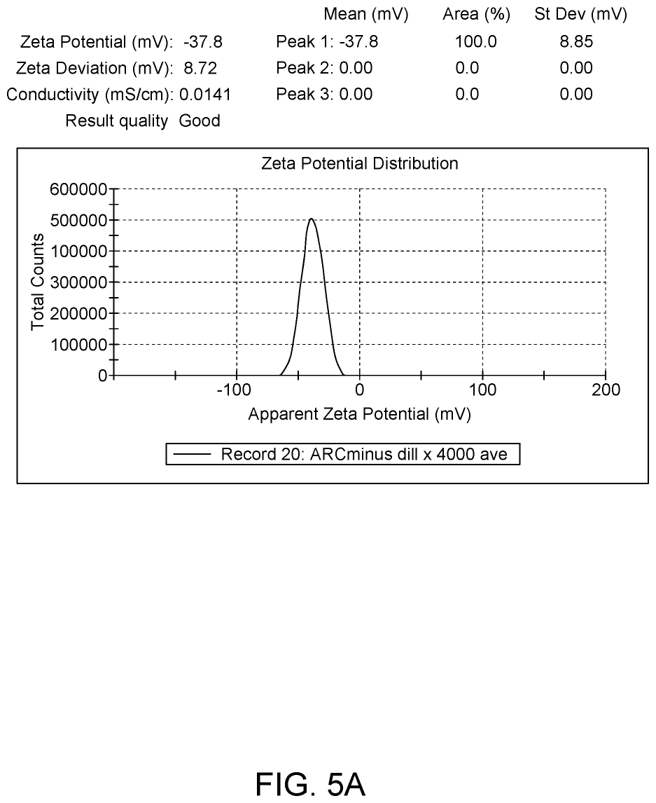

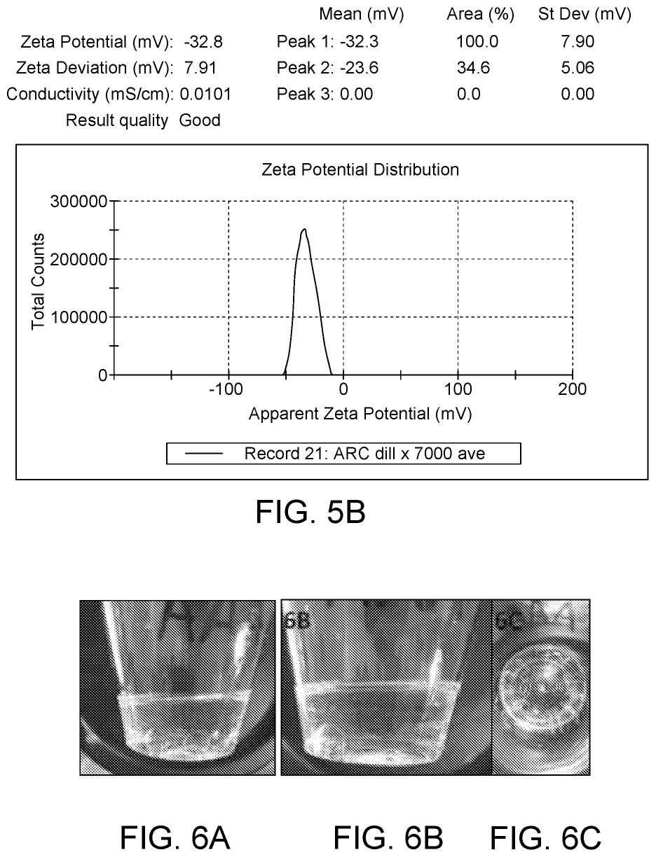

FIG. 5A shows the z-potential of in the mineral extract composition (without Se/Zn).

The data (FIG. 5A) show a single major peak of z-potential of −37.8 mV. This value indicates the existence of repulsive forces that enable a stable dispersion in the presence of the minerals extracts without the Se/Zn trace elements.

FIG. 5B shows the z-potential of mineral extract in an embodiment of the present composition with Se/Zn.

The data (FIG. 5B) show a two main peaks of z-potential −32.3 mV and −23.6 mV. These values indicated the reduced z-potential in comparison to the mineral extract composition without Se/Zn. These figures (FIGS. 5A and 5B) confirm the existence of coacervation induced by the Se/Zn system as observed in the comparative microscopic imaging of Examples 4-8.

### Example 9

This example evaluates the concept of trace elements selenium and zinc mixture as used in the present emulsions and conditions. Example 4 shows the effect of the trace elements selenium and zinc mixture on a water-in-oil mixture (without emulsifier). Briefly, a 0.2% w/v solution of trace elements selenium and zinc in doubled distilled water (DDW) was prepared. Then a water-in-oil mixture was prepared by mild mixing (e.g., magnetic stirring for 10 seconds) of the trace elements solution with paraffin oil (volume ratio 1:10).

FIGS. 6A, 6B and 6C show very poor dispersion of particles. This example indicates negligible effect of trace elements on the oil constituents.

### Example 10

This example evaluates the effect of the nonionic surfactant Tween 80 on coacervation in a water-in-oil mixture. Briefly, a water-in-oil mixture was prepared by mild mixing (e.g., magnetic stirring for 10 seconds) of the doubled distilled water (DDW), nonionic surfactant polysorbate 80 (Tween 80) with paraffin oil (volume ratio 1:1:10).

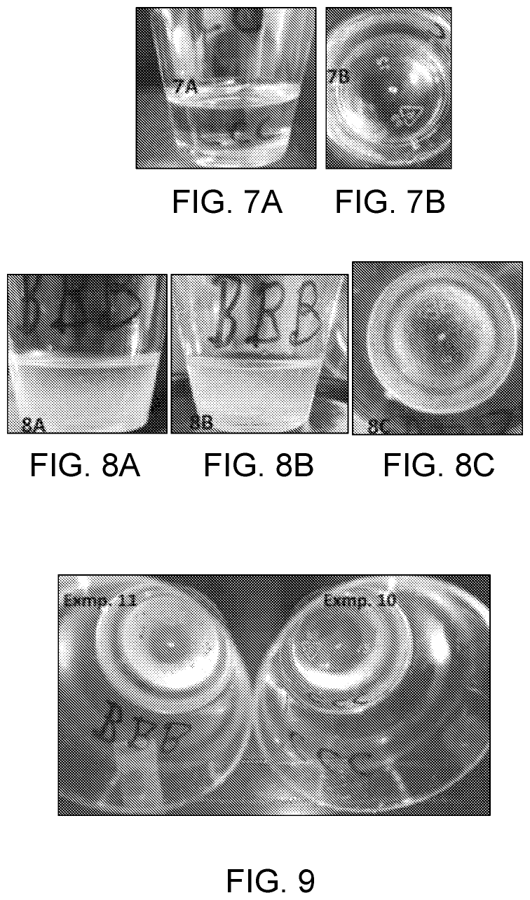

FIGS. 7A and 7B show very poor emulsification of water in oil by Tween 80. As Tween 80 is an oil-in-water surfactant, the observed poor emulsification of water-in-oil was expected.

### Example 11

The same w/o emulsion of Example 10 (water, oil and surfactant 1:1:10) was prepared by mild mixing (e.g., magnetic stirring for 10 seconds). But the DDW was replaced with the same volume of the trace element selenium (Se) and zinc (Zn) 0.2% w/v solution in DDW.

FIGS. 8A, 8B and 8C show a significant dispersion of droplets in comparison to Examples 9 and 10. This example indicates the interaction between the trace elements selenium and zinc with the nonionic surfactant and oil interface. This indicated that trace elements induce coacervation in a nonionic surfactant based dispersion.

### Example 12

This example is a comparative observation of Example 10 in comparison to Example 11 under the same experimental conditions. The sample from Example 10 shows the result of mixing the water in oil and Tween 80 as a surfactant (ratio 1:10:1). The sample from Example 11 shows the significant emulsification which is greater than that of Example 10 with the addition of the trace element solution.

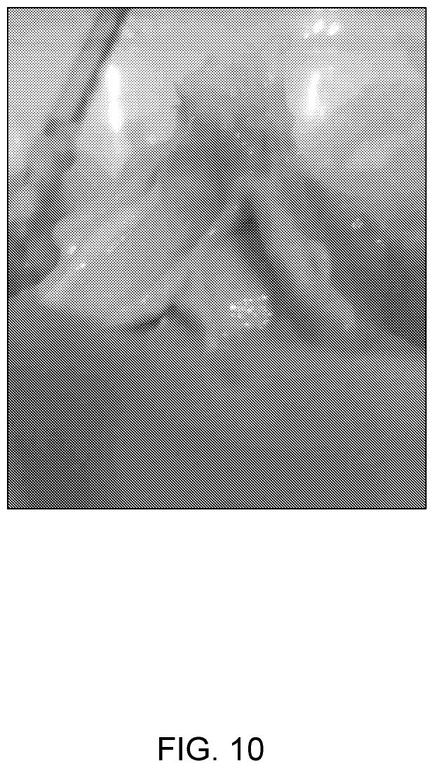

This comparative presentation (FIG. 9) indicates that selenium (Se) and zinc (Zn) increased the emulsification of the surfactant.

### Example 13

The example describes an embodiment of the present composition with 4% (w/w) essential oils and 16% (w/w) ethanol of the total weight of the composition.

The photo (FIG. 10) shows the semi-solid texture of the cream base with no indication of reduced viscosity nor any disruption of the cream base consistency.

### Example 14

This example shows a set of clinical cases where patients were treated with embodiments of the present composition. The results show the skin protective effect of an embodiment of the present composition.

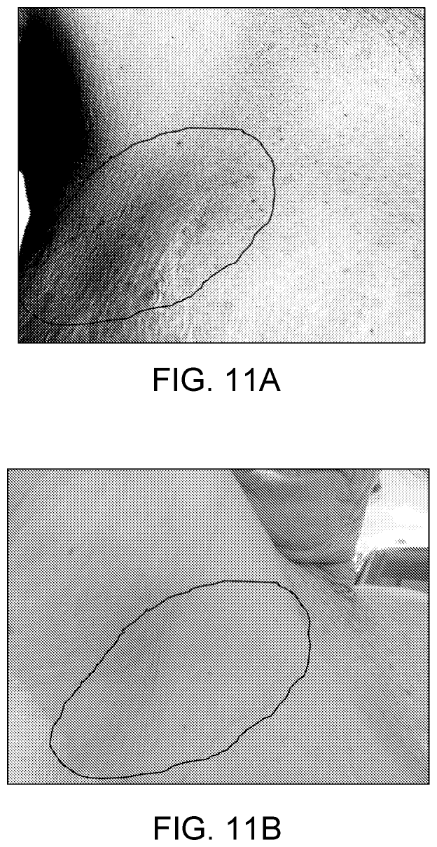

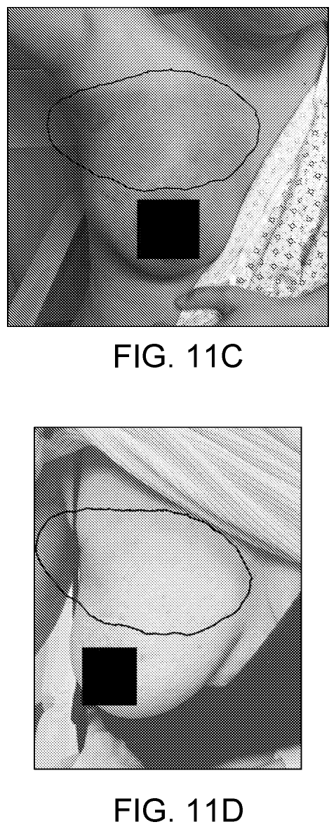

FIG. 11A is a photo depicting the damaged skin of a female patient with breast cancer treated with radiation therapy.

The marked area in FIG. 11A depicts the erythema and desquamation foci at the site of radiation application.

FIG. 11B is a photo depicting the same patient of FIG. 11A after 1 week of daily topical application of an embodiment of the present composition.

The marked area in FIG. 11B shows significant recovery of the skin damage after a 1-week treatment period.

FIG. 11C is a photo depicting the damaged skin of another female patient having breast cancer treated with radiation therapy.

The marked area in FIG. 11C depicts dark skin pigmentation—no desquamation using of an embodiment of the present composition from the beginning of radiation therapy. Only light pigmentation change observed at this early stage of the treatment course.

FIG. 11D is a photo depicting the same patient of FIG. 11C after 4-week of radiation therapy cycles treated daily by an embodiment of the present composition.

The marked area in FIG. 11D depicts significant healthy skin with no indication of skin damage, nor pigmentation or dryness.

### Example 15

This study used comparative electron microscopy cryo-SEM assessments of the controlled coacervation induced by trace elements in an embodiment of the present composition (Example 2).

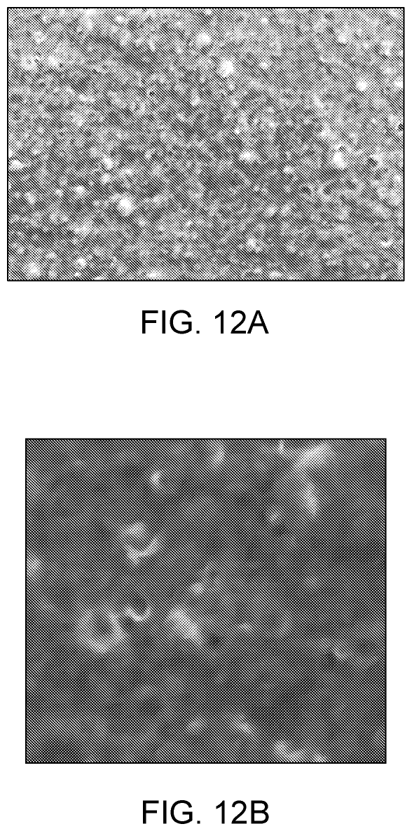

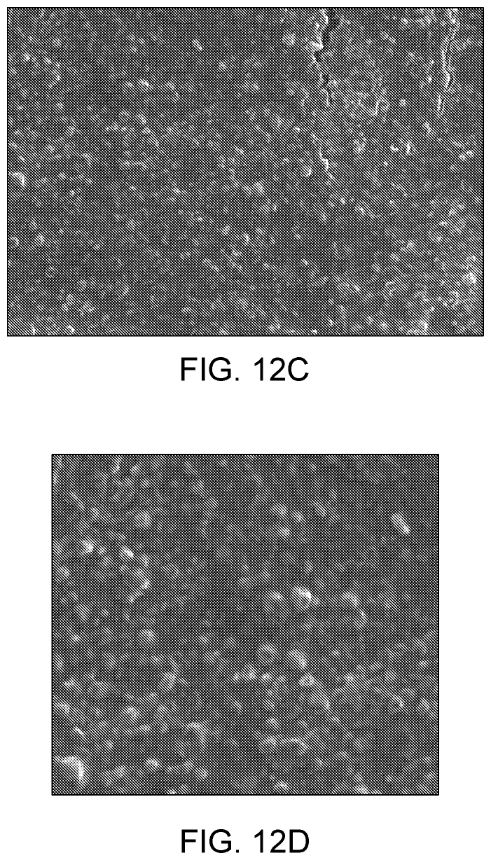

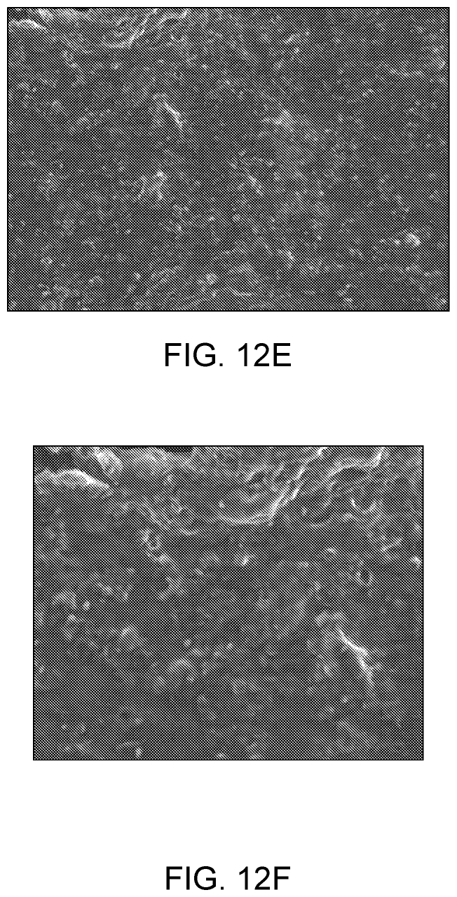

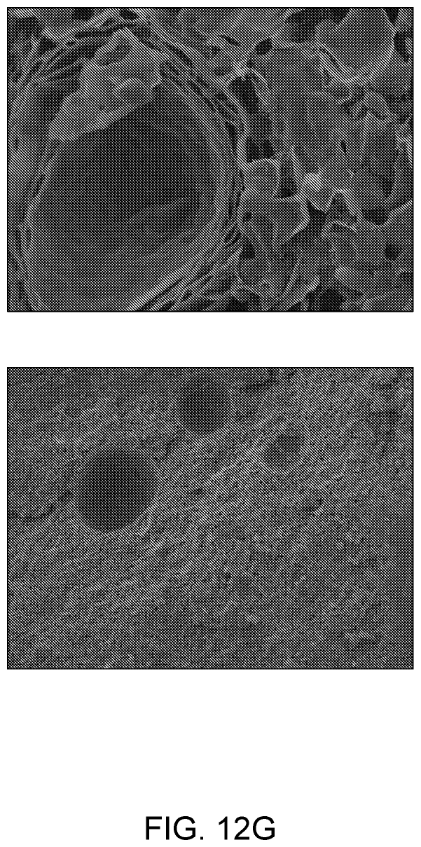

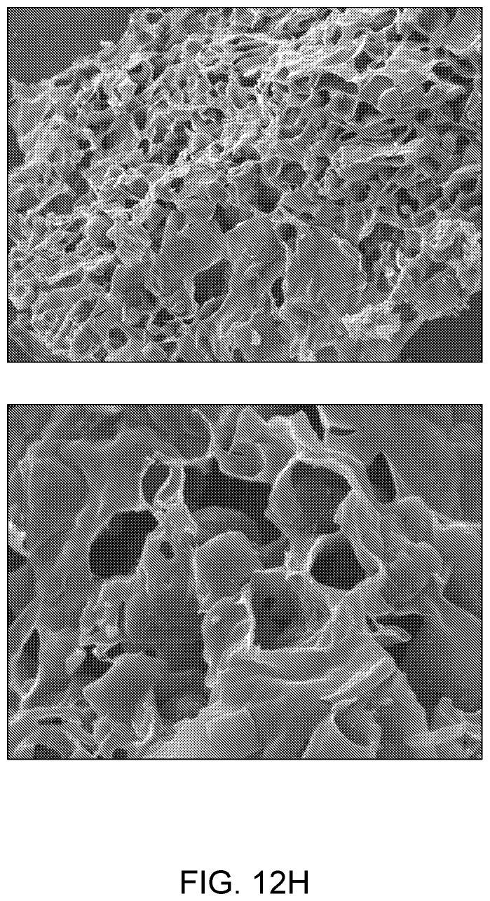

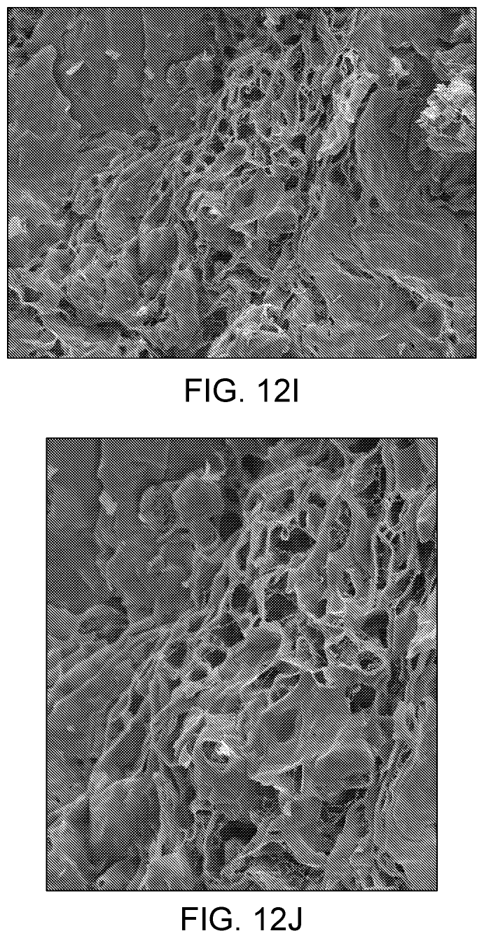

FIG. 12A shows the morphology of the composition with the mineral extract (Example 1) but without selenium (Se) and zinc (Zn). FIG. 12B shows an enlarged portion of FIG. 12A.

FIG. 12A and FIG. 12B show the dispersion morphology of the composition (as described in Example 2). In this study, we added selenium (Se) and zinc (Zn) to the blend of the extracted minerals (to a final concentration of about 0.02 wt %) as depicted in Example 1. Note that the dispersion comprises lipid vesicles embedded in the cream matrix. However, the observed morphology of the dispersed vesicles was characterized by relatively poor spherical shape, notably the resolution view was poor due to the lack of distinctive structures. The presence of the black pores indicated residual water that were not encapsulated in the vesicles and not entrapped in the matrix. This correlated with the relatively lower viscosity of this cream base.

FIG. 12C shows the morphology an embodiment of the present composition with only selenium (Se) and zinc (Zn) as described in Example 2. FIG. 12D shows an enlarged portion of FIG. 12C.

FIG. 12C and FIG. 12D show the cream matrix and the dispersion morphology of the composition (as described in Example 2). Note that FIG. 12C and FIG. 12D show the microscopic view of the composition comprising only selenium (Se) and zinc (Zn) without any other minerals used in Example 1. The figures show a plurality of dispersed lipid vesicles with defined spherical shapes embedded in the cream matrix. This observation supports the differentiated effect of selenium (Se) and zinc (Zn) as effective coacervation inducing agent versus the relatively poor effect of the extracted minerals of the samples of FIGS. 12A and 12B.

FIG. 12E shows the morphology of an embodiment of the present composition with only selenium (Se) and zinc (Zn) as described in Example 2, except that the concentration of the trace elements was increased to 0.06 wt %.

FIG. 12F shows an enlarged portion of FIG. 12E.

FIG. 12E and FIG. 12F show the cream matrix and the dispersion morphology of the formulation (as described in Example 2) except that the concentration of selenium (Se) and zinc (Zn) was increased to 0.06%. These observations indicate a significant effect of the increased selenium (Se) and zinc (Zn) concentration. When the concentration of selenium (Se) and zinc (Zn) increased from 0.02% to 0.06% by weight of the total weight of the composition, the cream matrix and the dispersion lost their stability. As can been deduced from FIG. 12E and FIG. 12F, the increased concentration of (Se) and zinc (Zn) affected significantly the dispersion uniformity and morphology of the vesicles. The number of vesicles per matrix area reduced dramatically and most vesicles have poor shape and morphology with less spherical structure. The data indicate that the desired coacervation and stable dispersion of (Se) and zinc (Zn), as depicted in FIGS. 12C and 12D, is optimal within a narrow concentration range.

FIG. 12G shows more examples of the morphology of the composition with 0.06 wt % selenium (Se) and zinc (Zn).

The upper panel of FIG. 12G shows another example of a large size multilamellar vesicle with a size of about 15 μm, as observed in the composition with the 3-fold higher selenium (Se) and zinc (Zn) concentration (i.e., 0.06 wt %). As shown in FIGS. 12E and 12F, although the increase of selenium (Se) and zinc (Zn) concentration (from 0.02 to 0.06 wt %) caused a significant loss of dispersion consistency, the ability of these trace elements to produce vesicles of multilamellar geometry was preserved. As the image shows, the core of the large size vesicle has a significantly increased diameter and was surrounded by an ordered lamellar morphology. This supports the coacervation-induced multilamellar morphology of the vesicles, although fewer (much larger) vesicles were produced when the selenium (Se) and zinc (Zn) concentration was increased to 0.06%, as shown in the bottom panel of FIG. 12G. The upper panel of FIG. 12G shows that the surrounded matrix of the vesicle did not have the same ordered texture as seen in Example 5.

FIG. 12H highlights the observed differences of the matrix. FIG. 12H shows more examples of the cross-section morphology of the composition with 0.06 wt % selenium (Se) and zinc (Zn). FIG. 12H show the cross sections of the cream matrix of the composition with the increased concentration of selenium (Se) and zinc (Zn) to 0.06 wt %. In comparison to the cross-section image of Example 5 (cross-section of the composition), the data show that the increased Se/Zn concentration elicited significant effects on the cream matrix. The effects included a significant disordered orientation and distribution of the matrix lamellas which support the observed thermodynamic sensitivity of the composition to selenium (Se) and zinc (Zn) concentrations.

FIG. 12I shows the effect of reduced Se/Zn concentration by 50% (to 0.01 wt %) in the composition. FIG. 12J shows an enlarged portion of FIG. 12I.

FIGS. 121 and 12J show the effect of reduced concentration of selenium (Se) and zinc (Zn) from 0.02 wt % in the composition disclosed in Example 2 to 0.01 wt %. The images show that this reduction also affected the integrity and consistency of multilamellar geometry and morphology of the matrix of the cream base in comparison to the well-defined morphology depicted in Example 5 (with 0.02 wt % Se/Zn). This is another indication of the concentration thermodynamics of the trace elements in the composition.

### Example 16

This example depicts further evaluations of the physical effects elicited by the trace elements selenium (Se) and zinc (Zn) in nonionic surfactants-based emulsions. This Example studied oil-in-water emulsions, while Examples 9-12 addressed the effect in water-in-oil emulsions.

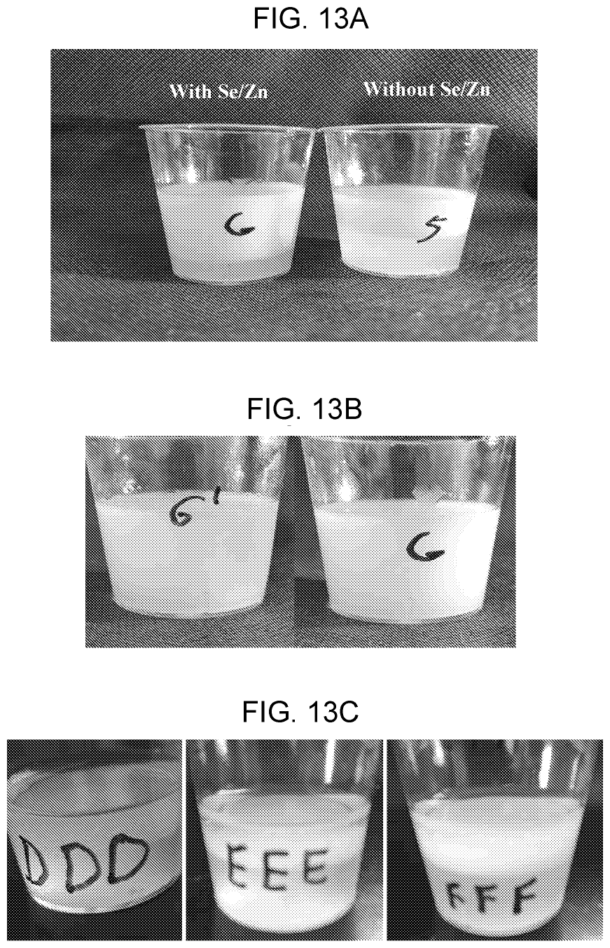

An oil-in-water emulsion was emulsified with the nonionic surfactant Tween-80. The sample marked on the right of FIG. 13A (Sample 5, marked with “5”) contained 40% (w/w) paraffin oil, 50% (w/w) DDW, and 10% (w/w) Tween 80. The sample on the left in FIG. 13A (Sample 6, marked with “6”) contained 40% (w/w) paraffin oil, 50% (w/w) DDW, and 10% (w/w) Tween 80, with the water phase also including selenium (Se) and zinc (Zn) in a final concentration of 0.01% w/w.

FIG. 13A shows the effect of Tween 80 as a nonionic surfactant in an oil-in-water emulsion 1-hour post emulsification. In this test we indicated the significant increase of emulsified phase when selenium (Se) and zinc (Zn) were added to the emulsion. This data indicated that the trace elements synergized the effect of the nonionic surfactant which may be due to coacervation.

FIG. 13B shows the effect of other minerals on the emulsification capacity of FIG. 13A after vigorous stirring.

This example (FIG. 13B) shows the effect of selenium (Se) and zinc (Zn) of Sample 6 depicted in FIG. 13A in the presence of 0.04 wt % NaCl (Sample 6′). The data indicate that in the presence of minerals such as NaCl, the emulsification capacity was reduced. Sample 6′ was a turbid/opaque dispersion rather than milky emulsion as Sample 6. This example indicates that additional minerals may reduce, and/or interfere with, the coacervation mediated by selenium (Se) and zinc (Zn) alone.

FIG. 13C shows additional comparative assessments of the physical effect of selenium (Se) and zinc (Zn) and their concentrations on nonionic surfactant-based emulsion. The test sample DDD was used as a control and contained 40% (w/w) paraffin oil, 50% (w/w) DDW and 10% (w/w) Tween 80. Samples EEE and FFF contained 0.03 wt % Se/Zn (Sample EEE) or 0.18 wt % Se/Zn (Sample FFF) in its water phase, respectively, in addition to 40% (w/w) paraffin oil, 50% (w/w) DDW and 10% (w/w) Tween 80.

The data show the effect of the trace elements selenium (Se) and zinc (Zn) on the emulsification mediated by the nonionic surfactant Tween 80 in an oil-in-water emulsion. The three samples, DDD, EEE and FFF, were prepared under the same condition. In Sample DDD, a mild and partial, but steady, emulsification was observed. This outcome is correlated with the use of Tween 80 as a nonionic surfactant for oil-in-water emulsions. We used a relatively high load of oil phase (the weight ratio of the lipids to the aqueous medium was 4:5) in order to observe mild (but not maximal) emulsification of Tween 80 as a baseline for reliable evaluations of the trace elements effects. As indicated from Samples EEE and FFF, the addition of selenium (Se) and zinc (Zn) in a final concentration of 0.03 wt % and 0.18 wt % surprisingly increased the emulsification capacity by a significant manner in this repeated study. Considering the lack of interaction between selenium (Se) and zinc (Zn) with the oils, as depicted in Example 9, these data support the conclusion that the selenium (Se) and zinc (Zn) induced coacervation may be associated with the nonionic surfactant mode of emulsification.

### Example 17

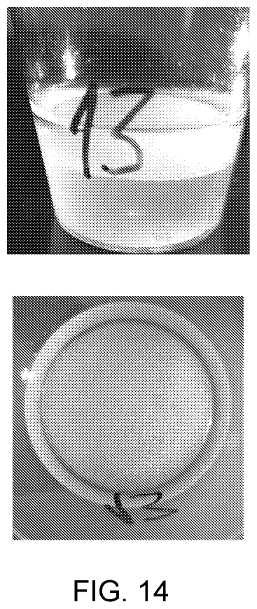

FIG. 14 shows oil-in-water basic emulsions emulsified with the nonionic surfactant Tween-80 as an exemplary surfactant. Sample 13 contained 30% (w/w) paraffin oil, 60% (w/w) DDW, and 10% (w/w) Tween 80. The water phase included selenium (Se) and zinc (Zn) in a final concentration of 0.04 wt % and NaCl 0.16 wt %.

This example shows the reduced and limited effect of emulsification of the nonionic surfactant in the presence of selenium (Se) and zinc (Zn) with NaCl. Moreover, the addition of NaCl which increased to 0.16 wt % inhibited the coacervation induced by selenium (Se) and zinc (Zn). Note that the ratio of lipids/aqueous medium was reduced to 1:2 in order to enable optimal conditions for the Tween 80 to emulsify the phases. However, the increased concentration of NaCl exceeded the threshold of coacervation and induced a salt-out effect of the surfactant as indicated by the significant foam at the image taken from the upper side of the sample (the bottom photo). These data indicated that the selenium (Se) and zinc (Zn) was thermodynamically sensitive to the presence of other minerals in the composition.

### Example 18

Table 4 shows the comparative viscosity evaluations of the same tested groups as depicted in Tables 1 and 2. The objective of the comparative study of viscosity was to evaluate the effects of the trace minerals Se/Zn on the rheology profile of the composition. The measurements of viscosities of the different formulations were performed under the same experimental conditions using the digital rotational viscometer model NDJ-8S, MRC laboratory-instruments, Essex, UK (measurement range 10-2,000,000 m Pa·s, accuracy ±1%). The composition of Example 2 (Group 1) in comparison to the composition without the trace elements Se/Zn (Group 2) and the composition with mineral extract, without Se/Zn (Group 3).

TABLE 4
comparative viscosities

| Parameter | Group 1 | Group 2 | Group 3 |
|---|---|---|---|
| Viscosity (Pa · s) | 118 ± 2.9 | 48 ± 2.0 | 76 ± 5.0 |

Pa · s-pascal-second.

The data show the significant higher viscosity of the Group 1 composition (with Se/Zn), compared to the Group 2 composition (without the addition of trace minerals) and the Group 3 composition (with the mineral extract). The data indicate that the observed comparative morphologies of vesicles and the multilamellar matrix of the composition as depicted in Example 6 correlated with the rheological effect of Se/Zn on coacervation which increases the viscosity of the composition.

### Example 19

Table 5 shows the comparative evaluations of the SPF (sun protection factor) of the same tested groups as depicted in Tables 1 and 2 in comparison to a commercial SPF-15 sunscreen (positive control). The measurements of SPF of the different formulations were performed under the same experimental conditions using a validated (ISO 9001, 2015) digital SPF apparatus model SPF290AS, Solar Light Inc. Glenside, Pa., USA. The Group 1 composition (with Se/Zn) was compared to the Group 2 composition (without the addition of trace minerals) and the Group 3 composition (with the mineral extract). In this SPF test, the Group 1 composition (with Se/Zn) as disclosed in Example 2 was tested as wet (Group 1a) and dry (Group 1b) conditions. The commercial approved sunscreen carrot oil SPF-15 (Sun & Care, Dead Sea, Israel) was used in this test as for positive control.

TABLE 5
Comparative SPF

| | Sample name | | | | | | | | | |
|---|---|---|---|---|---|---|---|---|---|---|
| | | | | | | | | | Positive Control | |
| | Group 1a | | Group 1b | | Group 2 | | Group 3 | | SPF-15 | |
| | Sample batch | | | | | | | | | |
| | 24 Jun. 2020 | | 25 Jun. 2020 | | 25 Jun. 2020 | | 25 Jun. 2020 | | 24 Jun. 2020 | |
| | Value | STDV | Value | STDV | Value | STDV | Value | STDV | Value | STDV |
| SPF | 0.65 | 0 | 0.65 | 0.01 | 0.69 | 0.02 | 0.65 | 0.01 | 35.7 | 11.72 |
| UVA/UVB | 0 | 0 | 0 | 0 | 0 | 0 | 0 | 0 | 0.702 | 0.01 |
| ratio | | | | | | | | | | |
| Boots Star | 0 | No | 0 | No | 0 | No | 0 | No | 3 | Good |
| Rating | | Claim | | Claim | | Claim | | Claim | | |
| (2004) | | | | | | | | | | |
| UVA I/UV | 0.82 | High | 0.84 | High | 0.89 | High | 0.84 | Hight | 0.81 | High |
| Ratio | | | | | | | | | | |
| Max % T | 1.16 | | 1.47 | | 4.27 | | 1.32 | | 33.96 | |
| COV | | | | | | | | | | |
| Critical | 0 | 0 | 0 | 0 | 0 | 0 | 0 | 0 | 377.5 | 0 |
| Wavelength | | | | | | | | | | |
| Curve Area | −17.32 | 0.09 | −17.57 | 0.27 | −16.3 | 0.84 | −17.81 | 0.16 | 141.35 | 14.45 |
| UVA PF | 0.69 | 0 | 0.68 | 0 | 0.69 | 0.01 | 0.68 | 0 | 18.7 | 6.34 |
| Erythema | 0.66 | 0 | 0.66 | 0 | 0.68 | 0.01 | 0.66 | 0 | 16.15 | 3.75 |
| UVA PF | | | | | | | | | | |

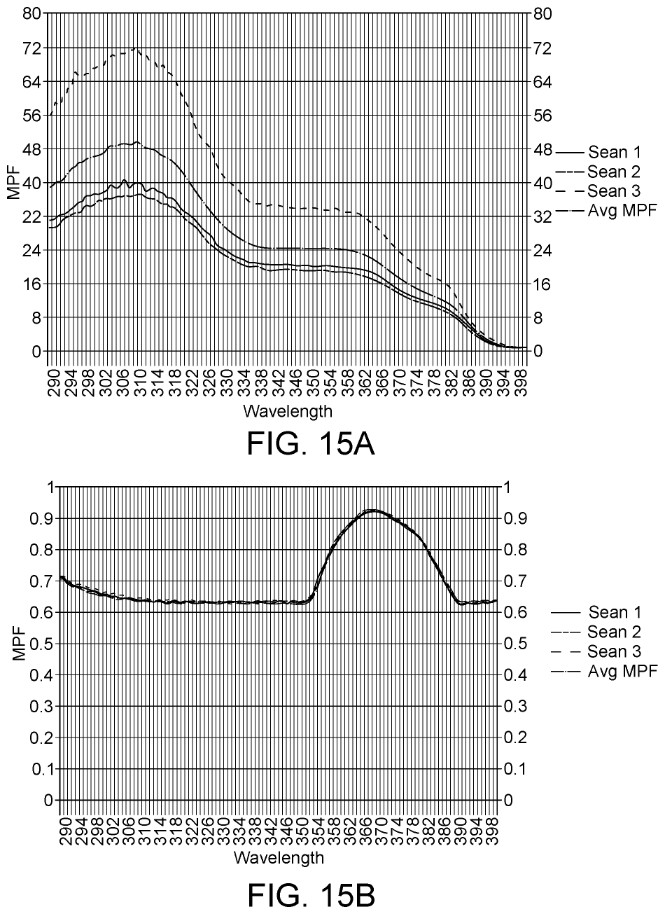

The data show that the Group 1 composition (with Se/Zn) as either wet or post drying (15 mins before radiation as the standard test procedure), as well as the Groups 2 and 3 compositions, exhibited significantly low SPF levels in the range of 0.66-0.65. While under the same experimental condition, the positive control carrot oil SPF 15 sunscreen showed SPF of 16.15. These data indicate that the composition did not absorb radiation. This property of the composition shows that pretreatment of the skin by an embodiment of the present composition before radiation therapy will not interfere with the radiation efficiency. FIGS. 15A and 15B show examples of SPF data as a measurement of absorption spectra of the tested samples in this study.

### Example 20

Example 20 used an ex vivo model to evaluate the photoprotection effect of an embodiment of the present composition on human skin exposed to UVB irradiation, in comparison to a commercial SPF-15 sunscreen as a positive control. The biological markers for epidermal cell viability and the response of proinflammatory mediators (such as IL-8) indicate the ability of the tested composition to protect the human skin against UVB-induced erythema, edema, and inflammation. Positive results indicate the ability of the tested composition to protect epidermal cells against UV-induced photoaging and inflammatory effects.

The photoprotection of human skin grafts (abdominal skin of healthy human subjects) was evaluated following the pretreatments with the tested groups. The photoprotection profile of an embodiment of the present composition was compared with the positive control under the same experimental conditions. Human skin grafts were pretreated with either an embodiment of the present composition (e.g., the Group 1 composition) or a commercial SPF15 sunscreen, and then were exposed to 450 mJ/cm² UVB irradiation for 15 minutes. After UVB irradiation, the skin grafts were incubated under sterile conditions of 37° C., 5% CO² for 24 hours until the tests of cytokine responses, or for 48 hours for the viability assay.

TABLE 6
Comparative photoprotection evaluations

| | | | UVB |
|---|---|---|---|
| Sample | Treatment | Conditions | (450 mJ/cm²) |
| 1 | Sham Control | No treatment | No radiation |
| 2 | Control | No treatment | UVB |
| 3 | Positive Control | Commercial sunscreen | UVB |
| | Pretreatment | (Carrot Oil SPF-15 Sun & | |
| | 0.25 hours | Care, Dead Sea, Israel | |
| | before UVB | | |
| 4 | Tested Composition | Group 1 composition | UVB |
| | Pretreatment | (ARC) | |
| | 0.25 hours | | |
| | before UVB | | |

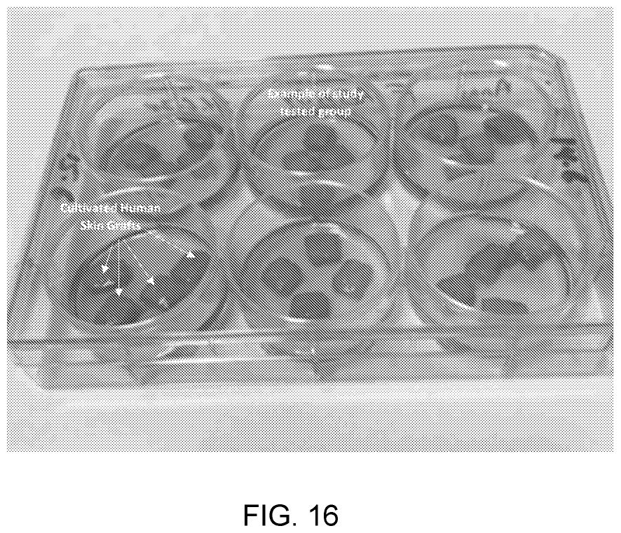

FIG. 16 shows a photo of a tested 6-well plate with cultured human skin grafts in the growth medium as evaluated in the photoprotection study.

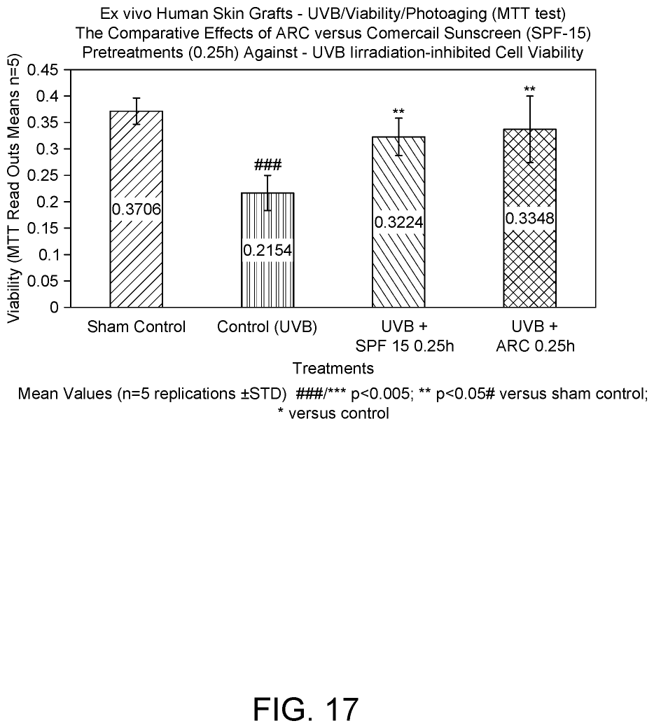

FIG. 17 shows the comparative photoprotection results of epidermal viability of the human skin grafts treated with the Group 1 composition (ARC) compared to the commercial SPF15 sunscreen (positive control) under the same experimental conditions. The viability of human epidermal skin cells was evaluated by the MTT ((3-[4,5-dimethylthiazole-2-yl]-2,5-diphenyltetrazolium bromide) assay at 48 hours post UVB irradiation. All tested groups were performed in replicates of n=5 per group. The data shows the significant effect of UVB irradiation on skin viability (control versus sham control), a reduction of sham control viability levels by 42±3% with significance of p=0.002. Under the same experimental conditions, the Group 1 composition (ARC) and the positive control sunscreen protected the skin against UVB induced cell death (viability loss) by 67% (p=0.01) and 76% (p=0.02), respectively.

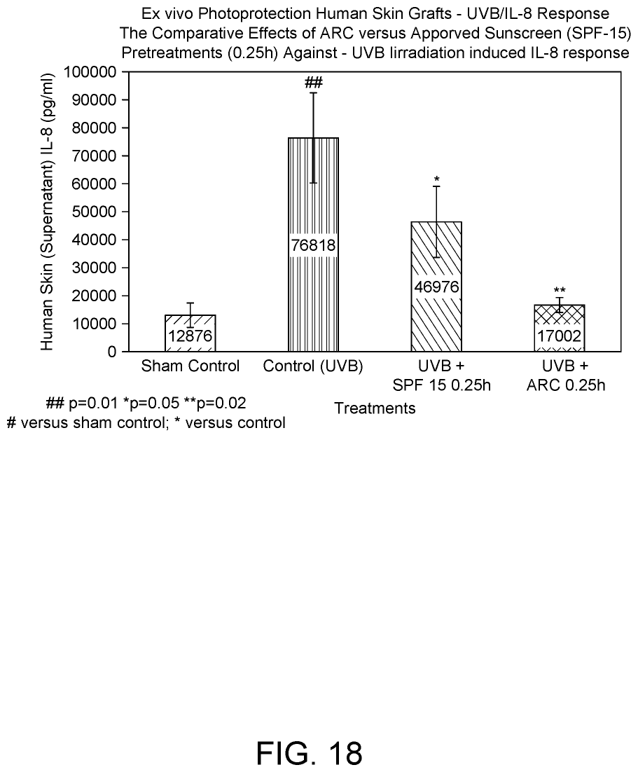

FIG. 18 shows the comparative photoprotection results of interleukin 8 (IL-8) of the human skin grafts treated with the Group 1 composition (ARC) compared to the commercial SPF15 sunscreen (positive control) under the same experimental conditions. The data shows that UVB irradiation induced IL-8 release significantly at 24 hours post UVB irradiation. All tested groups were performed in replicates of n=3 per group. UVB-irradiated human skin grafts released about six times higher levels of IL-8 compared to the sham control (p=0.01). Pretreatment of the skin graft by the positive control (commercial SPF-15 sunscreen) inhibited the UVB-induced IL-8 release by about 40% (p=0.05). Under the same experimental conditions, the Group 1 composition (ARC, an embodiment of the present composition) inhibited the UVB-induced IL-8 release by about 80% (p=0.02). The data demonstrate that the present composition can provide about 2-fold of potency against IL-8 release compared to the positive control (the SPF-15 sunscreen).

### Conclusions

The present composition demonstrated significant skin protection effects. The high water entrapment efficiency of the multilamellar matrix of the composition helps protect human epidermal cells against UV-induced photoaging and inflammatory effects. This effect protects the skin against UVB radiation without the use of chemical UV filters. The data emphasize the efficacy of the aqueous oil-in-water multilamellar matrix to hydrate the extracellular microenvironment of the skin layers exposed to radiation and to protect the human skin against tissue damage and inflammation. Moreover, the results of the SPF test show that the present composition can protect patients against radiation dermatitis without interfering with the radiation efficacy to treat cancer.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description is offered by way of illustration only and not as a limitation.

1. A composition comprising an aqueous medium, lipids, and at least one nonionic surfactant, wherein the composition comprises lipid-based vesicles dispersed in the aqueous medium, wherein the lipid-based vesicles are unilamellar and/or multilamellar and have an aqueous core, wherein the lipids comprise at least an oil, and wherein the composition comprises at least one trace element having a concentration ranging from about 0.0001% to about 0.1% by weight relative to the total weight of the composition.

2. The composition of claim 1, wherein the composition comprises at least one trace element having a concentration ranging from about 0.015% to about 0.025% by weight relative to the total weight of the composition.

3. The composition of claim 1, wherein the composition comprises at least one trace element having a concentration ranging from about 0.018% to about 0.022% by weight relative to the total weight of the composition.

4. The composition of claim 1, wherein the at least one trace element is selenium (Se), zinc (Zn), or a combination thereof.

5. The composition of claim 4, comprising about 0.02% selenium (Se) and about 0.0002% zinc (Zn) by weight relative to the total weight of the composition.

6. (canceled)

7. The composition of claim 4, substantially free of minerals other than Se and Zn.

8. (canceled)

9. The composition of claim 1, wherein the at least one nonionic surfactant is a polyethoxylated saccharides derivative, a polyethoxylated sugar alcohol, a sugar ester of fatty acid, a sugar alcohol of fatty acids, an emulsifying wax, a fatty alcohol, a pegylated lipid, a silicone oil, a silicone oil derivative, a glyceride, a polysaccharide, derivatives thereof, or combinations thereof.

10. The composition of claim 1, wherein the lipid-based vesicles have a mean size ranging from about 0.1 micrometers to about 10 micrometers.

11. The composition of claim 1, having a hydrophilic-lipophilic balance (HLB) value ranging from about 10 to about 14.

12. The composition of claim 1 having a hydrophilic-lipophilic balance (HLB) value of about 12.

13. The composition of claim 1, wherein the weight ratio of the lipids to the aqueous medium is about 1:1.5 or about 1:1.7.

14. The composition of claim 1, wherein the weight ratio of the at least one nonionic surfactant to the lipids is about 1:1 or about 1.5:2.

15. The composition of claim 1, having a z-potential ranging from about 1 mV to about −60 mV.

16. The composition of claim 1, having a z-potential ranging from about −20 mV to about −40 mV.

17. The composition of claim 1, wherein the lipids further comprise at least one wax.

18. The composition of claim 1, wherein the composition comprises a multilamellar matrix.

19. The composition of claim 1, wherein the aqueous medium has a pH ranging from about 5 to about 6.

20-25. (canceled)

26. The composition of claim 1, further comprising at least one anionic surfactant.

27. The composition of claim 26, wherein the at least one anionic surfactant is a fatty acid salt.

28-30. (canceled)

31. A method of preventing or reducing skin damage in a subject in need thereof, the method comprising: topically applying to the skin of the subject an effective amount of the composition of claim 1.

32.-49. (canceled)
